(19) United States
(12) Patent Application Publication
(10) Pub. No.: US 2025/0128090 A1
(43) Pub. Date: Apr. 24, 2025

(54) UV PATHOGEN ERADICATION

(71) Applicants: Israel Nissenbaum, Brooklyn, NY (US); Asher Baum, Brooklyn, NY (US); Mitchell J. Bogart, New Haven, CT (US)

(72) Inventors: Israel Nissenbaum, Brooklyn, NY (US); Asher Baum, Brooklyn, NY (US); Mitchell J. Bogart, New Haven, CT (US)

(21) Appl. No.: 18/820,189

(22) Filed: Aug. 29, 2024

Related U.S. Application Data
(63) Continuation-in-part of application No. 18/097,367, filed on Jan. 16, 2023.
Application No. 18/097,367 is a continuation-in-part of application No. 17/244,860, filed on Apr. 29, 2021, now Pat. No. 11,554,187.
(60) Provisional application No. 63/535,427, filed on Aug. 30, 2023.
(60) Provisional application No. 63/017,407, filed on Apr. 29, 2020.
(60) Provisional application No. 63/044,641, filed on Jun. 26, 2020.
(60) Provisional application No. 63/077,003, filed on Sep. 11, 2020.
(60) Provisional application No. 63/118,638, filed on Nov. 25, 2020.
(60) Provisional application No. 63/149,611, filed on Feb. 15, 2021.

(51) Int. Cl. A61N 5/06; A61L 2/10; A61N 5/067

(52) U.S. Cl. CPC A61N 5/0624; A61L 2/10; A61N 5/0603; A61N 5/067; A61L 2202/11; A61N 2005/0605; A61N 2005/0606; A61N 2005/0609; A61N 2005/061; A61N 2005/0626; A61N 2005/063; A61N 2005/0643; A61N 2005/0644; A61N 2005/0651; A61N 2005/0661; A61N 2005/0663; A61N 2005/0667

(57) ABSTRACT

Manual, automated and mass methods and devices, for enhancing the effect of UV light disinfection (at UV wavelength of 200 nm to 340 nm) of transmitted UV light on or in humans and devices by at least one of the steps of: a) increasing controllable output power of a UV emitting device, including using high power UV lasers or direct placement of UV light sources, b) increasing the efficiency of UV disinfection effect with the placement of the UV emitting device including by use of side emitting fibers with uniform output and distant placement directed at surgical sites, otoscope type handheld devices directing UV light into infected orifices, biofilm disruption, fluorescent marking of pathogens and c) providing protocols for various enhanced pathogen eradication applications including tissue clearing to increase depth of penetration, use of aspiration needles for access to pathogens, site disinfection to increase cancer remission and implant in situ disinfection.

This application takes priority from U.S. Provisional Application 63/535,427 filed Aug. 30, 2023 and is a continuation-in-part of U.S. patent application Ser. No. 18/097,367 filed Jan. 16, 2023 which is a continuation in part of U.S. patent application USSN 17/244,860, filed Apr. 29, 2021, now U.S. Pat. No. 11,554,187, issued Jan. 17, 2023, which in turn takes priority from provisional patent applications: 63/017407, filed Apr. 29, 2020; 63/044641, filed Jun. 26, 2020; 63/077003, filed Sep. 11, 2020; 63/118638, filed Nov. 25, 2020; 63/139294, filed Jan. 19, 2021; and 63/149611, filed Feb. 15, 2021, the disclosures of which are incorporated herein by reference thereto.

### FIELD OF THE INVENTION

This invention relates to methods and devices for the enhancement of pathogen eradication with UV light primarily by remote means and particularly relates to the eradication of pathogens by UV light in or on medical instruments and to the non-traumatic eradication, by UV light, of pathogens within or on human or animal bodies. In particular, this invention further relates to UV transmission pathogen eradication parameters, components, and applications with preparation and operational feedback of effectiveness and more particularly to transmission with collimating lenses, laser UV sources, pathogen site and transmission device preparation, coupled with eradication feedback in various sterilization or sanitization settings.

### BACKGROUND

In said parent patent applications, devices and methods were disclosed which effectively eradicated pathogens such as viruses, bacteria and cancer (defined as a pathogen), primarily by means of remote application of UV light. Such UV light was specifically exemplified as coming from an LED UV light source (though other light sources were described), to infected areas such as in biopsy channels of endoscopes and within a human body by means of a transmission medium, in particular, with an embodiment of a fiber optic transmission medium. The specific pathway of the eradication was by means of a disruption of DNA/RNA of the pathogens (or cancer cells) on a molecular level without or with minimal physical trauma to the organ or healthy cells harboring the cancer, with a complete or almost complete elimination of pathogens or a significant reduction of pathogen presence sufficient to alleviate untoward conditions. Such eradication effectiveness was however constrained, in efficient utilization, by considerations, such as application time and power of applied UV light, lack of effective feedback, skill required for application, and limitations encountered with minimized depth of penetration of UV light.

In further discussions hereafter, the term “pathogen”, as used herein, is defined as referring to noxious and/or toxic elements having DNA/RNA, subject to disruption, and specifically refers to viruses, bacteria, cancer (though not technically a pathogen but being toxic and having DNA/RNA), mold and mildew, and other micro-organisms. Also included in the term “pathogen”, for purposes of the invention disclosed herein, are cells and tissues which are healthy in themselves but are excessive or in improper biological locations, with such excess, providing noxious conditions. In addition, healthy cells which are presently benign, but which may become toxic, such as by being adjacent to non-healthy cells such as cancer cells or viruses and bacteria, which may spread, are similarly defined herein as being “pathogens” or “pathogenic” for purposes of being disrupted with UV light. Microorganisms, as well as small insects, susceptible to being affected by UV light by lessening their noxious activity are further included within the term “pathogen”.

The term “eradication”, as used herein, refers to the deactivation of noxious and/or toxic elements by disruption of DNA/RNA contained in these elements with breakage or altering of the bonds of the helix structure of the DNA/RNA components. The terms “disinfection”, “sanitization” and “sterilization”, as used herein, are used interchangeably as referring to a medically acceptable cleaning. The term “sterilization”, when so indicated, is an FDA recognized term for total or log 6 or greater reduction of pathogens.

The terms “transmission” and “transmission media (medium)” of and with UV light, as used herein, and as used in the parent application, refer to the acting as a vehicle to transport a sufficiently acceptable power-output of UV inactivating energy to a pathogenic, or possibly pathogenic, site, including viruses, bacteria, cancer and other known or otherwise defined pathogens. The terms include fiber optics, liquid light guides, light pipes, and optical lenses and lens systems specifically configured to provide a length distance which acts similarly to a fiber in transmitting disruptive UV light from a UV light-source via collimating (with essentially parallel light rays), focusing or other lens, lens combination, or lens and fiber combination, and transports the light along its collimating or focal length to be directed to the pathogen site. This is in contrast to simple transparent media, such as glass or clear plastic panes, as protectively used such as in flashlights which simply allow a light pass-through. The term “angle of acceptance” is used generically with respect to all transmission media and refers to the angle or direction for light and particularly UV light of a designated wavelength to be successfully introduced into the transmission media for the transmission thereof. “Energy” (Power×time) and “Power” (Energy/time) are used interchangeably herein. The term “focusing” in this, and the parent application, has the meaning, depending on context, of technical optical control with a lens or the like, or a generic meaning of directing transmitted light into small areas. The term “collimating” means the substantial maintaining of a light beam diameter over a relatively pre-determined short distance with maintaining of power, until gradual dissipation. The term “reasonable period of time” which is used with a period of UV light pathogen eradication is a function of the particular application of the pathogen eradication, the reasonable economic or medical exigencies and severity of pathogen infection. Thus, disinfection of non-biological devices such as endoscopes may be longer (depending on requisite processing requirements) than required for disinfecting invasive medical procedures. Reasonable time for medical procedures such as cancer treatment may be longer than for virus or bacteria treatment. In general, without specific limitation, reasonable time should not normally exceed a half hour for each pathogen eradication site, though normally much less. Unless otherwise indicated, the term “site”, as used herein, refers to a single pathogenic or suspected pathogenic area exposed to UV light. The term “full”, as used herein, is to be construed as being “substantially full” unless otherwise indicated.

The prior applications have shown and exemplified the reality of effectively transmitting UV-C light with power sufficient to eradicate pathogens in a reasonable time in various settings of difficult to reach or inaccessible areas or in accessible sites, in effective concentrations. More effective uses and devices, with greater number of applications and practical operational feedback remain to be developed and implemented.

### SUMMARY

In view of the above, it is an object to provide methods and devices for enhancing the effect of UV light disinfection (at UV wavelength of from 200 nm to 340 nm and most particularly in the UV-C range of 250 nm to 285 nm, with a peak effectiveness for DNA disruption at about 265 nm and 220 nm being considered safe for surface use only, but with essentially no depth of penetration) and pathogen eradication by at least one of the steps of (among others): a) increasing controllable output power of a UV emitting device, b) increasing the efficiency of UV disinfection effect with the placement of the UV emitting device and c) providing protocols for various enhanced pathogen eradication applications.

It is a further object to provide such controlled increased power with a laser UV light source, having controlled characteristics, to within the DNA/RNA disruption UV wavelength (most particularly centered at 266 nm though other wave lengths within the UV-C range such as 261 nm and 254 nm are available and useful) and power range, for eradicating pathogens, before onset of traumatic ablation or surgical transection of tissue and to optionally use minimal ablation levels for removal of dead pathogens or cancer cells, with at least some diffusion-of-light expedients. Though lasers with UV transmission media are known, they have not, as far as is known, been generally used for sterilization and have been configured and utilized for ablative purposes such as surgical transection, general etching or for spectroscopic analysis.

It is a further object to provide UV and specifically UV-C light emitting lasers, with relatively high output power, which are coupled to, or are capable of being coupled to, a transmission medium, such as transmitting fibers or other transmission media including lenses and lens systems, and which are of effective, but low-cost construction, rendering them useful in general sanitization or sterilization applications. Small beam diameter UV laser output also enables even lower output power to be effectively used in very flexible fibers with diameters in the range of 100 microns or less. The small beam diameter also enables the use of adjuvant illuminating light carrying fibers and the inclusion of camera elements for direct viewing or recording of disinfection sites.

It is another object to utilize controlled, increased power of a UV laser light, and of very high-powered UV LEDs or similar high power light sources, in conjunction with a side-emitting optical fiber with a gradient of light output and substantially unform power output along its side. Side emitting fibers coupled to UV LED power such as disclosed in US Patent Publication 2021/0122667 have shown killing of pathogens. However, this publication discloses a relatively low power LED UV light source with a microwatt output requiring well in excess of 30 minutes for pathogen eradication and with only about 8 inches of side emission pathogen killing power—decreasing with distance from the UV light source). This is a very inconsistent unpredictable output rendering it difficult to provide effective pathogen disinfection in reasonably predictive times. However, with the vastly increased UV output power, a full-length fiber (typically 500-600 mm in the biopsy channel of a bronchoscope) or one of another appropriate length, can be made to be effectively suitable for full insertion into the biopsy channel of an endoscope with uniform power outputs in the multiple milliwatt range. Emission of UV light from proximal to distal end thereof is sufficient to rapidly kill pathogens in its exposure area with resultant full channel disinfection and greater predictability in pathogen eradication effect. With modification, as described hereinafter, an entire side emitting fiber may be made with an essentially uniform UV light side emitting output at relatively high pathogen killing power.

It is a still further object to increase the efficiency of UV emitting devices, by utilizing a lens transmission medium for relatively short-range UV light transmission particularly collimated transmission (or with optical transmission focusing or a combination of collimation and focusing) with pathogen disruption in partially accessible human or animal body areas such as in or adjacent to respiratory, alimentary or excretory orifices. Alternatively, the collimated (and/or focused) UV light pathogen disruption may be utilized in surgical procedures with body incisions and for protection against common pathogens such as MRSA (particularly during surgical procedures), and SSIs which are difficult to remove, even with antibiotics or common sterilization expedients. UV light, because of its DNA/RNA disruption is effective against any pathogen containing DNA/RNA including the ones resistant to other inactivation and sterilization methods.

It is an object to provide non-intrusive bathing of surgical sites before, during and after surgical procedures, with directed and pathogenic clearing UV light, to maintain an acceptably sterile surgical site with prevention of MRSA staph infections and possible sources of sepsis, not readily eliminated by normal disinfection procedures.

It is an object to provide collimating lenses with parameters to concentrate and extend reach of UV-C light, as transmission media, with a more consistent and uniform pathogen eradicating power in applications requiring only short distance-maintained power levels. The collimating lenses are, for example, embodied as lenses in penlight or otoscope-type or other devices for short distance pathogen eradication, including treatment of oral or nasal bacterial infections, hand sanitization, head lice combing, CPAP tubing sterilization, reduction of refrigerator bacteria pathogenic growth, cutting board and food preparation site sanitization, and the like. Since collimated light substantially maintains power density over the length of the collimation, UV light from a source may be positioned at a distance (such as out of the way of a surgical procedure) while maintaining pathogen killing effectiveness.

Relatively short range but uniform and stable UV-C energy output through a collimating lens or lens array systems, for a predetermined distance prior to divergence, finds application in sterilization devices such as in sterilization boxes for medical devices, instruments or tools, etc., particularly during surgical procedures to enable such tools and instruments to be quickly sterilized to be re-used during surgery. These devices, instruments and tools come in all shapes and sizes and contours making them difficult to uniformly and effectively sterilize. Short range multi-directional light serves to maximize effective UV sterilization light impingement. Though gradual divergence occurs at an optically measurable distance from the light source, based on lens dimensions and parameters, sufficient concentrated UV-C energy may be effectively positioned in the diverging UV-C light for an additional distance.

It is understood that collimation “transport” of light is the carrying of light with substantially uniform pathogen killing power over a significant pre-designed distance range, with minimal divergence. Generally, “transport” is the directing of light (while not necessarily collimated) but should have a predetermined purpose or effect of directing or shaping the divergence of a beam. In contrast, light “travel”, even through transparent media such as plano-glass is non-collimating with substantially or virtually unrestricted divergence.

In another object, surgical sites may also be optionally bathed with selected other wavelength lights, with known parameters and procedures, to cause bacteria to fluoresce and be more easily and effectively targeted in real time in situ situations. This provides real time feedback with pathogen visualization and/or pathogen eradication effectiveness is provided to validate effectiveness and to enhance effective pathogen control and eradication.

It is yet another object to provide pathogen treatment protocols for enhanced pathogen eradication such as by including successive DNA/RNA disruption eradication of surface pathogens, followed by ablative removal of dead cells and tissues, to increase effective UV light penetration and reach, to underlying tissue.

In another object, depth of penetration limitations are also sidestepped with protocols involving surface treatments which facilitate prevention of surface pathogen spreading. Internal reaming formation and widening of access lumens is similarly possible with aspiration needle carriers of UV light transmission members. With access lumens being formed, expedients such as suction and/or washing may be viable for removal of dead cells. Dead cell removal may also be carried out by scavenging phagocytes but, while useful, this expedient may be constrained by long times necessary for effectiveness. Robotic or automatic repetitive operations according to a pre-mapped operational route is more effective with respect to properly blanketing DNA/RNA disruption sites.

Another object for increasing enhanced pathogen eradication is by rendering the pathogen site, such as cancer tissue or tumor site itself, more permeable to light penetration by means of known “tissue clearing” procedures, particularly such as by removal of light blocking pigments and light scattering lipids with selected solvents to increase transparency of the tissue, with resulting greater UV light penetration depth particularly useful for in vivo procedures.

It is still yet another object to provide protocols and structures for UV light to effectively disinfect surfaces of implants and implant leads in situ within a body without the need for surgical removal of the implants or their leads.

It is another object to provide pathogen treatment protocols for increasing the extent of cancer remission by surface UV treatment of excised cancer sites especially as part of a surgical procedure, and similarly useful in periodic treatments subsequent to surgery as well. Prophylactic prevention of cancers may also be effected by surface treatment of possible cancer sites such as the pancreas, stomach and bladder.

As another object for increasing power output for disinfection, under appropriate conditions, one or more UV LEDs or other small UV light sources can be directly positioned, within a sufficiently large volume of an insertion device, such as a catheter or biopsy channel of an endoscope. The movably contained small UV light sources such as UV LEDs are properly oriented for direct UV light impingement, on pathogens within the insertion device which, without the need for transmission media with their coupling losses and attenuations, provide increased UV light power.

Another object for increasing efficient use of transmitted UV light is with automated and with computer generated data which includes initial determination of actual available UV light power by means such as power integrating measuring elements (integrated with or separated from the UV light source).

In this respect, available UV power level determination is coupled with controlled minimum and maximum dosage level determinations based on application distance, type of pathogen, power levels and DNA/RNA disruption efficiency to provide an automatic repeatable level of effective UV light energy delivered. A study and presentation of the effect of deep UV light on pathogens by the IEEE of Tech Talk Semiconductors Optoelectronics on 16 Apr. 2020, entitled Ultraviolet-LED Maker Demonstrates 30 Second Coronavirus Kill presented by Seoul Viosys and authored by Samuel K. Moore, provides a rough basis for determining the parameters of UV light (wave-length, application distance, applied power and duration) needed to have a disruptive effect on pathogens. The study provided the results of the direct (not transmitted) output of a non-optimal 275 nm (compared to 265 nm) LED on viral cells (coronavirus) with the conclusion that the virus was effectively killed in 30 seconds, with an output power of about 20 mW (thus an energy of 600 mJ) at a distance of 3 cm and an output beam angle of 120°. As a result of the inverse square rule, closer distances dramatically increase effective applied power intensity. In a parent application, extensive prior art tables were cited with detailed descriptions of hundreds of virus and bacteria pathogens with the required amounts of UV light treatment for the eradication thereof. These and other tables may be used in determinations, whether manual or automatic to determine the requisite parameters for specific pathogen eradication on an ad hoc basis.

A still further object, with respect to non-biological disinfection applications, for increasing efficiency and reliability in utilizing UV light for pathogen eradication is by including controlled and timed UV surface application onto pathogen infected areas (or suspected infected areas) and preparatory treatments to enhance UV light impingement on pathogen infected surfaces, particularly with biofilm disruption.

A further object for increasing efficiency of UV light utilization is the providing of a feedback determination loop wherein appropriate UV light application for pathogen eradication is controlled by real time feedback of extent of pathogen eradication, as a function of degree of pathogen infections needing disinfection.

Yet another object for efficient UV light efficiency entails the automating of disinfection of pathogen infected surfaces with controlled movement of a UV light source along the surface at effectively determined rates, and controlled power level application and distance between light source and pathogens.

Another object is to utilize small diameter aspiration needles to carry UV light fibers to nearly any part of a human body in vivo to provide UV light disinfection treatment thereto, and to provide requisite preparatory materials such as for tissue clearing to such part.

Still another object for efficiently disinfecting items with en masse procedures is that of large scale automated sterilization of devices such as pipettes, hypodermics and the like which must be used in medically sterile condition.

Another object is that of adapting the devices and protocols or pathogen eradication and additional sanitization or sterilization applications including those in dental and surgical site settings.

It is understood that the above objects and objectives are merely illustrative and are not exhaustive or limiting in any manner.

As used herein, the terms “power” and “energy” are used interchangeably, in the effect of killing pathogens. More specifically however, Energy output is a (cumulative) or amount or dose of energy required to kill or inactivate a pathogen which is described with the units of Joules, or millijoules or micro-joules. Power is the simple power-output rate as emitted from light-sources with the units of Watts or milliwatts or micro-watts. Time is the duration of directed power-output emitted from light-source, when directed at pathogen or biological entity, to kill or inactivate pathogen or biological entity, and is used in the cumulative effect of power to provide a measure of total Energy applied to a pathogen or pathogenic site.

Biological entities which are targeted for eradication. include microorganisms generally, viruses, bacteria, mold and/or mildew, tumors, cancer, cysts, etc. but also include cells or tissues, not yet harmful, but which may become harmful at a later time, e.g., cysts or growths that may become malignant. Also included are small insects such as lice which may be disrupted from harmful activity be UV light treatment.

Embodiments of methods herein for enhancing the pathogenic eradication effect of UV light of a wavelength between 200 nm to 340 nm on a pathogen infected area, include methods comprising at least one of the following steps.

The controllable output power of a UV light source emitting device is increased and directed within a difficult to access pathogenic containing area or an area requiring concentrated UV energy. Either direct placement of the UV light source or directed transmission of UV light from the UV light source is provided into either area. The direct or transmitted light maintains sufficient power to substantially eradicate pathogens in the pathogenic containing area, with DNA/RNA disruption.

UV light is trained, with directed transmission, onto a surgical site before, during and optionally after a surgical procedure to maintain a substantially pathogen free environment during the surgical procedure. In another embodiment, UV light is directed and transmitted through and onto an infected orifice for the disinfection thereof.

To increase depth of penetration of UV light, the pathogenic infected site is made susceptible to significant UV light penetration with pathogen eradication thereof by;
rendering the pathogenic site more transmissive to UV light penetration;
utilizing UV light, pathogen eradication to form a pathogen eradicated barrier between the pathogen infected site and a non-pathogen infected site; or
rendering the pathogen infected site more susceptible to the significant UV light penetration with the repeated steps of pathogenic eradication of infected areas and removal of the pathogenic eradicated areas to expose additional pathogenic infected sites to the UV light.

### Increasing UV Applied Output Power

Generally, the invention, in one embodiment, comprises the increase of input and output UV light power, particularly of UV-C light from the generally low single and double digit milliwatt output power practically available from readily available UV LED light sources to triple digit milliwatts and higher output UV light power by means of lasers emitting light in the UV spectrum range. Lasers currently exist in the UV range of 266 nm and with output powers exceeding 1 watt. Such lasers are normally exceedingly expensive and are used primarily for high powered ablation purposes and in some cases for use in tissue transection, as a function of the available power. These lasers have been effectively indirectly coupled to optical fibers (direct coupling entails problems with high power fiber degradation), primarily for control in precisely placing output laser light in effecting ablation or transection cutting purposes, particularly for corneal reshaping and eye surgery. Because of their inherent characteristics and small beam diameters, they have not been generally considered, constructed or configured for disinfection purposes where broad areas are needed for any useful or effective disinfection. Accordingly, broad and scattered lighting sources such as direct LEDs and lamps have been almost exclusively utilized for such purpose. In the present application, the laser light characteristics of minimal beam diameter are required for proper coupling with transmission media such as optical fibers through an angle of acceptance to maintain power, but subsequently, the output from the transmission media is specifically spread out, widened, or diffused for increased contact with pathogens for effective widespread disruption. UV lasers, in particular UV-C lasers, such as existing 261 nm or 266 nm or other UV pathogen or cancer inactivating wavelengths, provide a greater energy power output for increased flexibility in pathogen eradication applications with greater effectiveness in shorter time periods. UV-C lasers, when properly optically coupled to a transmission medium, such as an optical fiber (as well as lenses and collimating lenses or devices containing UV light transmitting media), enable the effective controlled and guided introduction of UV-C light into difficult to access areas of the body, including various organ tissues and organs, such as the brain, breasts, lungs, pancreas and other body parts such as bones and blood. Heat sinking or other accommodations may be necessary for protection or practical use of the optical fibers with pulsed lasers generating high peak power.

A power output in the range of about 100 mW-200 mW, is believed to be a reasonable transition level between the traumatic destructive ablation and transection levels of a 266 nm laser and the lower levels of non-traumatic, molecular level UV-C DNA/RNA disruption, though the transition range may vary in accordance with UV light source parameters and pathogenic area properties.

Various parameters of laser light characteristics are involved in the appropriate transmission coupling of laser light with optical fibers. These include M² determination of beam quality (usually with values less than 1.5 being more useful for coupling efficiency) and beam diameter relative to fiber diameter and destructive heat considerations with high peak power lasers endemic with pulse lasers. Furthermore, good M² beam quality helps to allow more usable light to enter into the optical fiber, without having to further increase the peak power to maintain a desired output power through the optical fiber that needs to enter the fiber core.

A UV laser design configuration embodiment, with coupled fiber (or other transmission medium) should include expedients, if needed, to minimize effect of high generated heat due to prolonged or even short use of the coupled fiber. Continuous wave UV lasers, with minimized heat build-up, are a desired laser system for consistent and stable power outputs, however such laser are presently generally of higher cost, size, weight and bulk, and overall lower output powers. UV-C lasers may be configured with multiple pulsed or continuous wave, laser outputs and are configured to provide pathogen eradicating UV wavelengths (such as 261 nm and 266 nm) and may be further configured to be single frequency and with narrow linewidth. Such lasers, in some embodiments, include use of harmonic frequency conversion, starting with one wavelength and converting to 261 nm or 266 nm, or other UV DNA/RNA disrupting range wavelengths, by means of crystals, such as with BBO (Beta Barium Borate) and/or optically known crystals to achieve desired UV output-energy results.

Alternatively, a laser diode of UV DNA/RNA disrupting wavelength (typically UV-C in the range of 200 nm to 285 nm with an optimum disruption wavelength of about 265 nm) is coupled directly to a transmission medium, for distance transmission, such as with mirrors, optical lens, lenses, or optical-fibers, etc. with greater initial laser energy enabling a final effective pathogen eradication energy.

Frequency doubling, by converting a 525 nm, 532 nm, or other wavelength laser directly with a BBO crystal may be used for achieving 261 nm or 266 nm wavelength outputs, or other UV disrupting wavelength with either pulse or continuous wave UV energy output. A narrow line-width is preferred when frequency converting through a nonlinear crystal, such as a BBO crystal for optimal UV conversion efficiencies.

The optical fibers used with the lasers may be specially treated to avoid burn-out, particularly if used with pulsed lasers of momentary high power pulses. Side emitting fibers normally are not effectively usable for disinfecting medical device channels such as biopsy channels. Such fibers have been disclosed (such as in the above cited publication) as being used with relatively low power LEDs with very low power output in the microwatt region, requiring exceedingly long exposure time (minimum of a half hour and usually up to several hours). In addition, the power output along the sides of such fibers have varying power intensities which rapidly drop with distance from the power source, such that fibers of more than about 8 inches (about 200 mm) have little or no power output at the distal end, whereas endoscope biopsy channels generally have much greater lengths (e.g., 500 mm-600 mm for bronchoscopes) and such fibers cannot effectively reach and sanitize the full length of endoscopes. The higher power enables even the distal end of a side emitting UV transmitting optical fiber, to be effectively sufficiently long to access the full length of a biopsy channel, to emit sufficient output power to eradicate pathogens in a more reasonable period of time.

An additional benefit of using lasers is their ability to emit very small diameter beams, which can be effectively directed into small core optical fibers with diameters of 50 μm, 100 μm, or smaller. These smaller core fibers have greater flexibility which allows for a tighter bend radius, less chance for breakage, and more maneuverability inside a difficult or inaccessible area, including endoscope channels and other curved geometries, including inside the human or animal body. It is preferred that the lasers, used in conjunction with smaller core diameter fibers be of continuous wave configuration to minimize possible fiber damage which may be incurred from high energy peak power pulses common to pulsed laser designs. Alternatively, shielding elements such as glass sleeves may be used to minimize such fiber damage.

In an embodiment, the smaller core diameter optical fibers, usable with UV lasers, are employed by being inserted into hollow, fine, aspiration needles, with diameters of less than 1 mm. Fine aspiration needles commonly used in EUS (Endoscopic Ultrasound) are carried through endoscope instrument channels for insertion into the bile ducts of a pancreas to collapse and destroy cysts, including Intraductal Papillary Mucinous Neoplasm's (IPMN's), a benign cyst, and a possible precursor to cancer, preventing cancer from forming in the pancreas. As non-limiting examples of other embodiments of use, superficial, yet difficult to target or reach cancers or precursors to cancer, may also include, but are not limited to ovarian cysts, polyps, lymph nodes, and intestinal metaplasia in the gastric mucosa, commonly found in the antrum of the stomach lining.

In an alternative embodiment the UV transmitting optical-fiber itself is provided with a transmissive needle-like end-treatment for direct penetration and UV transmission into a tissue site.

It is also possible, with high power UV light sources such as the UV lasers, mentioned above, as well as UV LEDs with high power output and die size/fiber connection compatibility (and the like), to correct for the uneven UV light output of side-emitting fibers of the prior art and progressive exponential loss over distance. In a procedure similar to that known in the printing art (as “stochastic screening”, wherein in printing microdots of black ink are formed. In this innovation, laser-formed microdots of light permeating holes are formed herein in the cladding to a depth of the fiber core. These microdot holes are produced, with cladding transparency, at varying concentrations at points along the length of a clad fiber to control the degree of UV light output and power. More microdot holes are formed at the distal end of the fiber as compared to the proximal end, with a gradient of concentration of microdots decreasing toward the proximal end. As a result, with this configuration and sufficient UV power, the final light transmitted at the end of the fiber is configured to provide a compensating gradient of transparency whereby light, which is side-emitted through the laser microdot holes, has a reverse power output change, i.e., light output power at the fiber end close to the light source has the least power emission with the emission percentage increasing with distance from the light source. Thus, the strongest light emission percentage is at the most distant end of the fiber furthest from the power source and the weakest light emission percentage is at the proximal end of the fiber closest to the UV light source. With judicious arrangement, power output along each side portion may thereby be rendered substantially uniform or with predictable utilizable power outputs.

This re-ordering of light output power may be used to compensate for the otherwise increasing attenuation at the most distant end. With this arrangement, the entire length of the optical fiber may be made to have substantially uniform side-emission along its entire length. Though this entails loss of the fiber having the maximum possible absolute side-emission, it is otherwise advantageous such that the fiber can, for example, simply be placed within a pathogen infected area such as a biopsy channel and remain stationary without the need for significant longitudinal movement. The higher power UV laser light compensates for the areas shielded from side emission, to provide a useful and uniform (or predictable) side emitted light output power. This eliminates or minimizes fiber moving procedures such as timed fiber withdrawals or insertions with attendant dwell time uncertainties, as well as likely errors with manual manipulation. To compensate for loss of fiber core protection with the array of microdot addition of a protective polymeric, a UV-transmissive outer layer may be used to surround the fiber with its microdot holes.

In another embodiment, in non-biological areas such as in the instrument or biopsy channel of an endoscope (such as a 4 mm diameter colonoscope) of areal cross sectional area in excess of about 12 mm², output power may be increased by elimination of losses inherent with light collection and transmission, and UV LEDs of slightly smaller cross section dimensions (generally of lower power outputs) can be directly inserted into such areas, with full LED power (not attenuated by connection to a transmission medium) being available for direct pathogen eradication. Though the effectively smaller LEDs, suitable for direct insertion, are generally of lower power output, elimination of large transmission losses with the direct insertion with lower power UV LEDs, more than compensates for high power LEDs having large transmission power losses. UV LEDs on controller PC boards have been miniaturized to an extent wherein the diagonal dimensions of the UV LED with PC board is under 3 mm. They are accordingly directly placeable into larger instrument channels of endoscopes such as colonoscopes with large diameter instrument channels generally of 4 mm or larger. The LEDs in such configuration are most effectively arranged along the length of the channel with UV light being directed against the channel walls. A triangular configuration of three UV LEDs arranged to alternatively face the channel walls with a 120° angle of light emission serves to provide a full 360° angle coverage of the channel walls, for a complete disinfection. These arrangements are only exemplary, with varying number of LEDs and placements being possible, depending on available areas and volumes.

In a further area, of an embodiment with effectively higher UV output power, a transmission medium of an optical lens is utilizable in UV light application devices such as of otoscope type hand-held configuration which remotely directs collimated UV light over short distances directly into orifices such as nostrils or the mouth and throat, or in excretory areas even when positioned external to the orifice. Alternatively, incisions can provide access to internal sites within the body requiring disinfection. Transmission of light with collimating lenses is of higher efficiency with less transmission loss and more effective UV light power output. With such directed high UV power devices, surgery sites, including the patient and any surgical incision sites themselves may be safely and comprehensively sanitized from even the most difficult to remove MRSA staph pathogens with a quick site sanitization before, during and after surgery. The collimated UV light is directed from outside of the immediate surgical area, without interfering with the surgery, with light directed only at the immediate surgical area where operating personnel are wearing protective gloves and wherein directed UV light does not stray into unprotected areas. Disinfecting UV-C light normally has little depth of penetration and is a safe yet highly effective surface disinfection treatment against surface pathogens such as staph infection, MRSA and other SSIs. Incidence of sepsis is thereby readily reduced. Collimated light, in contrast to optically focused light, maintains a substantially constant intensity output of power/unit area over the distance of collimation, thereby effectively increasing the distance at which the light source may be positioned, without significant loss of delivered power.

Distance from the light source leads to increased divergence of the light, which proportionally counters the effect of UV disinfection or inactivation. Distance further attenuates the UV light energy, thereby decreasing energy intensity concentration, leading to longer pathogen inactivation times and possible uneven and inconsistent pathogen disruption. Collimation of UV light in the manner and with the structure described herein serves to lessen the divergence with concomitant removal or minimization of a distance variable, even though there may be no visible perception of the collimation of invisible UV-C light. Use of collimating optics to “transport” the UV light along a determined minimized divergence length, allows for a more predictable and stable preservation of UV energy output for a more consistent and more uniform inactivation of the pathogen, as defined, within the collimation ambit, regardless of any perceptible light.

Collimation distance is generally a function of lens diameter, with the greater the lens diameter, the greater the length of effectively uniform power and the greater the distance that the light remains substantially effectively collimated prior to increased divergence and dissipation. It is understood that divergence always occurs even from zero inches to 10 inches, etc. Collimating lenses, however, lessen the extent of divergence or slows it down whereby uniform effective pathogen killing power is retained for a greater distance. The greater the dissipation, the less power is effectively brought to bear against a pathogen, with the longer the time necessary for pathogen eradication, to the point of essential ineffectiveness.

In fact, a concern with longer inactivation times is that, while generally UV inactivating energy is cumulative when directed at pathogens, too long of exposure requirement times, with insufficient UV inactivating energy to destroy the pathogens in a timely manner, may actually enable pathogens to build up a certain resistance to the UV eradication. Extended time duration, which may be necessary, as a result of excessive distance between UV light and pathogens, may thus result in enabling resistance to UV eradication to occur in the absence of timely kill rates. As a result, rather than all pathogens being killed, some might mutate to harmful forms.

Use of the collimating structure disclosed herein lessens failure by removing the distance variable from the inactivation equation, with sufficient eradicating energy intensity remaining constant or relatively constant over the entire length of the collimating ambit with non (or minimal)-divergent light output for defined, usually short, distances. Because of different distances involved in sanitizing a contoured device, there is a disparity in sanitization times, with some areas being sanitized before others. Other areas may still be in the process of becoming sanitized when the UV-C light is prematurely shut off, leading to incomplete sterilization. The collimating structure and configuration described herein results in a more consistent pathogen deactivation, regardless of the distance position within the collimating ambit. Thus, the same or similar UV energy output is delivered to the entire pathogen site. For example, whether a pathogen site is 5 inches away or 8 inches away. (typical collimating distance with collimating lenses of up to 2-inch diameter but is not limited to the size of a 2-inch diameter lens), ensuring a more confident and predictable sanitization of an entire device. Effective, though increasingly divergent light may continue for an additional useful distance, dependent on retained concentrated energy and pathogen susceptibility to UV-C light.

In biological applications, small diameter LEDs may be directly placed within a body in proximity to pathogens with a collimating lens being used to extend the effective power and reach of the UV output within limited areas. For example, an instrument channel of an endoscope is provided with a collimating lens as an end cap and with a UV LED being directly inserted into the instrument channel to a fixed (or within a movable range) position of optical alignment with the collimating lens to provide a power-preserved collimated UV light output against the pathogen. Similar body insertion devices such as catheters may be used to such effect.

In another embodiment a comb-like transmission medium is provided that can be dragged thru someone's hair in order to eliminate lice or larvae. Alternatively, a UV light emitting penlight device is used with an comb-like attachment which separates hair.

In a further embodiment an optical fiber cable includes a 100 μm core fiber for the UV, a bundled fiber that can carry a camera sensor image, and an additional fiber that may carry white illumination for viewing. This will allow entrance into areas of the body, such as IPMN's, lymph nodes, or bronchioles, etc., with a real viewing capture capability and with optionally included UV for inactivation of problematic biological entities, including cancer. The bundled optical fiber assembly, if thin enough, could be inserted into an aspiration needle such as an EUS, to penetrate into inaccessible sites, including in the pancreas.

Examples of short-range applications for the transmission medium collimating lens or lens system described herein include:

Emergency/Medical disinfection environments with short distance applications:
Throat, nasal, cold, or ear infections, including the eustachian tube, etc.
Sores, blisters or wound infections.
Ulcers caused by diabetes or other disease.
Topical, but difficult to reach skin or surgical or surface cancers, e.g., where scabs may form.
Hand sanitization devices either separate from or incorporated into hand drying or blower devices.
Inactivation of drug-resistant pathogens including MRSA and to be used in preventing Surgical Site Infection (SSI).
Industrial sanitizing tools to clean surfaces or below surfaces, or difficult to reach areas.

Military environment:
Sterilization and/or inactivation of biological agents, on or off the battlefield.
Treatment of infected wounds or other infections.

General environment:
Sanitization of difficult to reach spaces, such as in bathrooms, kitchens, basements, attics, drains, ducts or pipes.

In a further embodiment, a UV-C emitting mechanism such as in a penlight configured device, is used and directed at pathogenic activity. In an in-vivo environment, such as application to a human or animal body, UV-C light is targeted against surface layers or tissue with proximate internal orifice applications (i.e., oral, nasal, aural or vaginal) having surface pathogenic accumulation. The vast majority of pathogenic incidence is as a surface condition with the serendipitous nature of UV-C light being ideal for safe effectiveness, with lethal pathogenic eradication effect but with limited penetration. Thus, surface pathogens are effectively targeted with minimal effect on underlying healthy tissue. LED or optical fiber end-treatments, whether positioned directly or adjacent to the affected site, may include additional transmission media such as collimating or other lens, lens systems, mirrors or light-guides, for beam directing and beam shaping purposes, e.g., use of an optical or transmission attachment that can transport and “shape” or “bend” the light at areas typically inaccessible such as with the pharynx which runs along a vertical axis and perpendicular to the mouth/oral cavity.

In an embodiment of a UV transmitting penlight, it is comprised of a camera sensor, along with fluorescence-stimulating LEDs (optionally with filters), to help identify pathogen activity, which may be activated with the fluorescence-stimulating LEDs. With some pathogens, even UV light triggers fluorescence to simultaneously provide a pathogenic target for the UV light, killing pathogens and as a feedback mechanism for gauging effectiveness of eradication. White, RGB, or natural cool/warm LEDs, such as used for general illumination may be included in other embodiments for light emission together with the other emitting wavelengths, or separately with a light-guide that carries the light towards the distal-end of the penlight.

In a further penlight embodiment, attachment elements are provided and configured which are snapped into place or affixed on top of the penlight device in order to control beam shape, size, distance and the like.

Additional embodiments of attachment elements of the penlight, include an otoscope-type configured “head” or “imaging head” attachment. This “imaging head” optionally includes a camera sensor imaging system with optional display, for further clarifying diagnosis, fluorescing pathogen activity, and effect of UV therapeutic delivery. Alternatively, the otoscope-type device is an integrated self-contained device.

In another embodiment, a penlight configuration is adapted to fit into an otoscope-type shell, wherein the penlight is converted to fit into the otoscope and wherein the otoscope handle, of greater diameter, is provided with an adjuvant battery supply to increase effective use time of the penlight/otoscope before recharging or replacement is necessitated. The terms, “penlight”, “otoscope”, or “otoscope-type device” are used herein to refer to any handheld or mounted devices or fixtures, which includes a UV light source with UV light transmissible media.

As referred to above, collimation of UV light is particularly effective with respect to hand or body sanitization or sterilization in the form of hand sanitizers, (particularly in hospital settings where rapid and complete sterilization is a must) with the UV-C light transmission via collimation being integrated with common hand-blowers, such as ubiquitously found in restrooms, airports, restaurants, parks, etc., to insure sanitization in addition to less stringent hand cleaning (and sterilization, where required). In such embodiments, a series of collimated UV-C LEDs are arranged to provide a 360° emission of UV light directed in a closed (to prevent UV-C from external leaking but not necessarily limited to this structure) slot sufficient for hand insertion. The collimated UV-C light is sufficient to completely sanitize hands inserted therein (even to the point of more rigorous sterilization) within several seconds (though longer times are included within the ambit of the invention, short time periods are more conducive to actual use). UV-C light having limited penetration, such hand sanitization affects hand skin to a minimal extent and less than that of strong soap and scrubbing. Alternative hand sanitizer embodiments may also include UV-C elements not being enclosed. When combined with a hand blowing dryer, heat generated by the UV LEDs may be routed and directed for the drying. Such a device may include an internal auto-shutoff timer to prevent extremely long exposures.

In an alternative embodiment, the number of collimated LEDs are minimized, with the LEDs being arranged to provide either a mechanical or electronic scanning of an inserted hand to effect sanitization with simple hand insertion.

In further embodiments, the collimated UV light is used during surgical procedures (at different times before, during and after) directly on surgical site areas, especially open surgeries such as abdominal and heart surgeries, which may involve the presence of a high incidence of drug resistant MRSA and SSI, which may lead to sepsis, organ failure, and death. Though MRSA and SSI are resistant to most drugs, they are nevertheless readily susceptible to DNA/RNA disruption of UV light for the eradication or minimization thereof during surgery with concomitant reduction in instances of sepsis or other untoward conditions. Patient safety and safety of the surgical personnel is serendipitously maintained since UV light is used essentially only as a surface “disinfectant” with minimal skin or tissue penetration and pathogens such as MRSA and SSI are almost always present as skin surface contaminants prior to skin incision during surgery. Surgical personnel are further protected with the use of surgical gloves and such gloves are constantly maintained in a state of sterilization because of the applied UV-C light.

Operating-table lamps, or other ceiling rigs or fixtures, are readily configured to include a collimated UV-C light with extended range sufficient to maintain energy with minimized divergence to the operating site. In a further enhanced embodiment, the collimated UV-C light is included within or adjacent to surgical light headgear to ensure sufficient UV-C disinfection or sterilization conditions at the operation site. Since UV light is essentially invisible it does not visually interfere with visual conditions at the operating site. Alternatively, collimated UV-C light may be separately directed at the operating site, whether automated (with timed emission) or manually, such as with handheld devices which may be used and positioned by a doctor, or other operating site personnel, over the patient. The distanced collimated light source is maintained out of a position whereby it does not, or, at most, only minimally, interferes with any surgical operations. In addition, the operating lamps may further include a camera sensor module with image and video capture and fluorescing feedback detection, as further described herein, to help highlight location of pathogen activity, prior to termination of pathogen with UV. The term “invisible” is used as a general characteristic of UV-C light (i.e., not in the visible wavelength range) and not as a requirement.

Transmission media for the transport of UV-C light, with maintaining of effective energy for sanitization, are any solid or liquid media such as optical fibers and light guides as well as collimating or non-collimating lenses or lens systems; total internal reflection (TIR) lenses; and mirrors, including parabolic and off-axis parabolic mirrors. These or other related optical media may be used by themselves or in combination with different media, to transmit a sufficient UV energy output for disruption of pathogenic activity of harmful or possibly harmful biological entities.

In an embodiment, at least one collimating transmission medium is fixedly coupled to the output of a UV-C light source in order to provide “safe transport” or “safe passage” of a more consistent, more uniform, and a more stable energy output, at an operational distance, in order to inactivate pathogenic or other harmful or possibly harmful pathogens, defined as biological entities (having DNA/RNA subject to UV-C disruption), i.e., cancer, cysts, etc., with consistent and timely deactivation results.

A further type of a biological entity includes the mucous or tissue lining of a sore or irritated throat. Although there is usually no infection, the tonsils and other tissues in the throat may swell. This can cause discomfort or a feeling that there is a lump in the throat. Ablation of a biofilm and/or impacted tissue with UV-C light enables new surface tissue regeneration with alleviation of some discomfort.

A collimating transmission medium, in an optical lens or lens system enables effective channeling and control, with lessened divergence of UV-C light, thereby maintaining a more consistent energy output with enhanced quality and effectiveness. Typically, though not limiting, such lenses may be of double convex structure with light directly entering a convex surface being refracted or bent to assume a substantially parallel output, for a distance of its focal length, before the light diverges and dissipates to relative ineffectiveness.

### Increasing UV Light Application Effect

A further embodiment for increasing penetration depth involves the rendering of the pathogen site such as cancer tissue or tumor more transparent such as with the in vivo in situ removal of lipids and or pigments within the pathogen containing tissue by use of known methods such as with suitable solvents and detergents in controlled amounts to prevent or minimize side effects. An example of a process of in vivo tissue “optical clearing” is described in iScience entitled “Physical and Chemical mechanisms of tissue optical clearing”, Volume 24, Issue 3, 19 Mar. 2021, 102178. Optical tissue clearing, particularly for in vivo purposes is an ongoing field of study with many improved, more efficient, faster and reduced side effect methods and materials being constantly developed for biological observation purposes.

In the described process, methyl benzoate (MBe) approved by the US FDA (21 CFR 172.515; FDA 2015) and the European Union (EU Regulation 1334/2008 & 178/2002; EU 2015) for use as a food-grade flavor ingredient and methyl salicylate (another food flavoring agent) are described as among chemicals which help render tissues substantially transparent. These compounds are non-toxic, or very mildly toxic, especially in small amounts, and they are used currently in foods, beverages and topical applications.

Though transparent rendering of tissues including tumors have generally been effected in dead animal specimens, this has been primarily done for examination of the specimens with instruments such as microscopes. Transparent renderings of tissues (tissue clearing) have recently been conducted in in vivo studies such as the above cited one, with minimal toxic effect, for the purpose of rendering animal (particularly mice) skin transparent, enabling direct viewing of ongoing biological processes. Use of an aspiration needle with controlled solvents and/other tissue clearing chemicals (determined with tumor or cancer site volume mapping) to minimally target only a tumor site (and some peripheral cells or tissue) with pigment or lipid removal solvents or detergents is of minimal concern because of the limited amount of the introduced solvents. Also, the generally minimal or non-toxic nature of the solvents and the fact that the tumor site is to be excised, in any event, further minimizes any safety concerns. It is further noted that the transparent effect may be only transitory with replenished lipids and pigments being formed in the cleared site.

As the pathogen site becomes more transparent (tissue inherently does not absorb light waves) the optical transmission depth of the DNA/RNA disrupting UV light becomes significantly enhanced. Pathogen disruption removal in conjunction with phagocytes and/or ablation procedures is more fully expedited. The increased transparency, in conjunction with greater applied UV power, can enable full pathogen sites such as cancer tumors to be effectively removed without surgery or common current highly traumatic and disruptive radiation such as X-rays and gamma rays, with minimal side effects and significantly less trauma. The UV fiber-containing needle can be inserted into the cleared tissue with radiation of UV light dependent on the end treatment of the fiber. Thus, a fiber with a full diffuser tip has a circular radiation and a depth of penetration on each side of the fiber in a 360° range. The needle moves the diffuser tip through the depth of the tumor whereby at least a substantial portion of a tumor can become bathed with pathogenic killing UV light. With a transparent tissue clearance allowing for fuller depth of penetration, the needle with fiber can be effectively used at a surface of the tumor and moved to various sites of the surface to insure fuller UV coverage of the entire tumor or cancer cell site. Substantially full side emitting fibers can further enhance the extent of pathogen eradicating UV light coverage.

It should be understood that current procedures for rendering tissues transparent require time, which may be several hours or days, and the procedure may be considered as a pre-op one, requiring return for the actual UV light treatment during a time window of maximum transparency. An article in Advanced Drug Delivery Reviews, volume 180, January 2022, 114037 validates the in vivo tissue clearing for introduction of treatments and viewing of results, with the describing of tumor tissue clearing for introduction of infrared radiation and heat therein for the treatment thereof.

Various protocols are included in embodiments of effective treatment, even without transparency pre-treatments, to enhance overall pathogen eradication despite lack of depth of penetration of typical UV light (generally on the order of about 40 microns).

A first protocol embodiment comprises the surface treatment by UV light of tumor removal sites such as the interior surface of a bladder. The effect of such surface treatment, especially if conducted over periodic times as determined by the specific type of cancer and/or aggressiveness, is the prevention or retardation of aggressive cancer from recurring in the original tumor or surrounding sites. This, in effect, serves to lengthen remission times without any surgical intervention, or in conjunction with known remission control procedures or surgical interventions. Specific examples of such surface treatments include inner bladder wall treatments, currently treated with BCG (weakened or killed tuberculosis bacteria) to provide a similar prophylactic treatment.

A second protocol embodiment comprises the firewall-like formation of a perimeter including the reduction or elimination of blood channels with nutrient supplies at the interface of existing cancer cells and healthy cells to substantially retard metastization of the cancer cells into healthy cells. The UV light is directed at the surface of the interface and below it to a predetermined effective level by means of penetrating hollow needles such as aspiration needles commonly used for taking biopsy samples exemplified by EBUS and EUS needles containing the UV transmitting fiber or transmitting medium.

A third protocol includes multiple insertions in a closely-spaced geometry of light emitting aspiration needles with UV fiber transmitters to provide a widening area of a cored lumen amenable to inner surface UV light treatment with successive removal of dead cells by either phagocytes or physical ablation with suction or washing.

### Implant Disinfection

In situ direct disinfection of surgically implanted medical devices, including pacemakers, medication dispensing implants or other electrical devices, prosthetic devices such as hip and knee replacements, and rods or other structural devices, is effected, in another embodiment, with minimal surgical intervention. Surgical removal for disinfection procedures and reimplanting can be obviated (with reduced surgical side effects and expense) with the in situ application of UV light onto the surface of the implanted medical device on its surface and at its interface with cell tissue. Some medical devices, such as pacemakers and medicine dispensing devices are implanted in pockets between a patient's outer skin and underlying muscle such as in the left shoulder area of a patient just beneath the skin. In a disinfection procedure, an access incision for laparoscopic (as a non-limiting example of a guiding insertion device) insertion of the UV carrying fiber is made, proximate to the implant. The UV fiber is inserted between the implant and the holding pocket to effect disinfection by applied UV light. In various embodiments, multiple insertions may be required. Configuration of the fiber end may be modified to provide a limited radial UV light output in just the direction of the implant. With the fiber being inserted directly through a skin incision, the fiber end may also be provided with a rigid transparent, extending or extended, surface-prying blade which serves the purpose of prying skin from implant, thereby facilitating insertion of the fiber end between pocket walls and the implant, and of movement of the fiber end relative to the implant surface for complete disinfection coverage thereof. The blade itself diffuses the UV light therethrough to increase contact surface area. Surgical instruments such as retractors may be used to separate the implant surface from the pocket walls for fiber insertion, but without removal of the implant.

For deeply implanted devices such as rods and hip and knee replacements endoscopic fiber carriers may be used, as described in the parent patent, for access to pathogenic (or possibly pathogenic) detected or suspected areas on such implants for disinfection. Because of the normally minimal depth of penetration of UV light, it can safely be used on accessible surfaces of implants for disinfection with minimal effect on surrounding tissues or cells.

### Instrument Disinfection

Disinfection of instruments with hard to reach or disinfect areas such as the biopsy or instrument channels of endoscopes was described in the parent application with the introduction of UV transmitting fiber through the channel at a rate sufficient to effect a substantially full sanitization. In embodiments herein such disinfection procedure can be enhanced (such as with shorter sanitization times or with the use of lower power) by physically pre-treating the inner walls of the channel such as with a thin absorbent string akin to a dental sponge-like “super” floss, a type of floss used to dislodge and/or remove any debris under and or around a dental “bridge” tooth with a circular brushing stroke or single, one-way, pull-through to blot up and/or disrupt any biofilm which may protect pathogens from the impinging UV light. Alternatively, existing disinfection procedures such as brushes which remove physical residue within biopsy channels but which are ineffective relative to pathogens may be utilized to enhance UV light pathogen disruption effect by disrupting light blocking biopsy films. It is understood that biopsy films develop and thicken over time and are normally not a significant concern with endoscopes which are disinfected immediately after each use before biofilm has formed to any detrimental extent. Occasionally, ineffective reprocessing procedures, however, leave pathogens in place, which does allow them to grow and build biofilms. Alternatively, a small rubberized collar around insertion strings or rods may similarly provide a “squeegee” biopsy film removal effect similar to window wiping. As a good prophylactic procedure, the endoscope channels should be internally exposed to sanitizing UV light prior to any endoscopic procedure.

### Full Endoscope Disinfection

Since endoscopes are primarily constructed of polymeric materials susceptible to degradation when exposed to UV light, the exterior of endoscopes have not normally been disinfected with UV light. In fact, studies have shown the detrimental effect of UV light on endoscopes. Though the channels of the endoscopes are lined with UV resistant PTFE, for purposes of reducing friction of inserted materials such as biopsy instruments, the exterior of an endoscope is fully exposed. Accordingly, the parent application describes the use of transmission media such as fiber optics to be inserted into such channels to direct UV light directly against the PTFE lining walls for disinfection purposes, without detrimental effect.

The studies showing detrimental effect of UV light on endoscopes, however, have specifically involved long term storage and exposure of the polymeric endoscope materials over extended periods of time of days. In accordance with another embodiment of the invention, the outer surfaces of endoscopes are exposed to UV light, and particularly UV-C light, for relatively very short durations of at most several minutes and, depending upon exposure distance and power output, for even as little as seconds per exposure site. This is well short of the time necessary for UV light degradation of polymers by breaking of the polymeric bonds, in contrast to the very short time for DNA/RNA disruption resulting from phosphorus linkage breakages, as used for pathogen disinfection. In an embodiment, a ring structure of diameter sufficient to accommodate the cross-section width of an endoscope, a 360° internally directed UV light is used to disinfect an endoscope lowered therethrough (or the ring moved along the length of the endoscope) at a controlled rate. Because of the disparity in dimensions between endoscope insertion tube, body and handle, several light emitting rings may be utilized for the separate, differently sized components of the endoscope to maximize UV sanitization effect by close proximity. Alternatively, a flexibly expanding and contracting ring, with maintained full 360° UV light exposure, may be utilized to provide the full endoscope body with UV exposure for disinfection.

Another embodiment comprises a sterilization box sized for enclosing typical devices and instruments, with the interior walls being lined with UV-C LED lights coupled with collimating lenses having overlapping areas of light impingement with multi-directional output providing quick, efficient, and reliable sanitization or sterilization effect. The UV-C light output from the collimating lenses results in more consistent energy density output to hit different curvatures and contours of a device, instrument, or tool with the same or similar UV energy output requirements to deactivate a pathogen in relatively similar times. Effective exterior sterilization times may be reduced to seconds whereby even devices having polymeric materials may be sterilized prior to any detrimental effect of the UV-C on the polymers. A supporting UV-C light transmissible support is used to support the instruments or tools and the box while allowing full sterilization.

As a characteristic of UV-C light applied with a collimating medium for sanitization or sterilization purposes, there is a controlled and more consistent and more uniform energy application at different distances along a collimation path with its relatively lowered divergence which provides a more controlled application, of relatively short duration UV-C light which takes into sterilization account, curvature and other surface irregularities and general dimensions of areas and devices being sterilized.

For rapid, non-degrading application, a 360° sanitization or sterilization cleaning is effected by surrounding surgical instruments or tools (supported on a UV-C light conducting transmitting medium support for complete exposure thereto) with minimization of UV-C bathing light to a level sufficient to effect sanitization or sterilization with a safety margin, within a pre-determined period of time. The terms “sanitization”, “disinfection”, and “sterilization” are used herein interchangeably, with “sterilization” being frequently used as the most thorough of the three, particularly in medical applications.

### Increased Efficiency of UV Light Application

In further enhancements of the disinfection processing of endoscopes and the like, a timed retraction mechanism may be utilized at the proximal end of an insertion fiber to move the fiber through an endoscope channel at a controlled rate sufficient to ensure more uniform and extensive UV light exposure to the interior walls of the endoscope channel for more effective pathogen eradication with significantly reduced opportunity for human error.

As a fail-safe method for ensuring disinfection, since disinfection provides no visible indications (procedures such as final ATP testing are helpful for certification of proper disinfection though such test are costly), UV light output is initially measured by a power integrating device such as an integrating sphere, an integrating non-spherical shape, or a calibrated UV intensity sensor, with the power output being calculated together with parameters of relative channel and fiber diameters to determine UV light distance application. If the pathogen is known, its susceptibility to being broken down by UV light can further be factored in, in a determination of the fiber dwell time and rate of retraction. In any event, large safety margins of time should be factored in to ensure that all pathogens likely to be encountered are eradicated.

A further embodiment of sanitization entails large scale simultaneous direct disinfection of devices requiring cleaning such as cylindrical pipettes, catheters, syringes or similar laboratory or medical equipment with multiple UV fiber insertions and UV disinfection on an assembly line. Arrays of the devices are processed on a continuing basis on a conveyor belt with openings of the device facing in a parallel single direction whereby a corresponding array of UV light conducting fibers are lowered simultaneously into the array of devices, for a duration sufficient to disinfect each of the devices, and then lifted to permit the conveyor belt line to move a fresh array of devices into position for disinfection.

Taking the prophylactic measure of using UV light to removal cyst and cancer precursor growths helps eliminate possible precancerous risks in a manner similar to removal of polyps during routine colonoscopies. Removal of IPMN cysts in the pancreas and intestinal metaplasia in the antrum of the stomach, minimizes irregular cellular activity in those areas and helps to eliminate cancer and metastasizing cancer risks. In addition, use of UV light to effect the cyst and intestinal metaplasia involves a minimally traumatic ablating experience on adjacent healthy tissue with reduced incidence of pancreatitis or similar side effects of prior art procedures. In such procedures, fluid is initially suctioned out of the cyst by means of the aspiration needle and the aspiration needle, with contained UV transmitting fiber is used to treat the interior of the cyst with DNA/RNA disrupting UV light to essentially destroy it. Once a cyst is removed or destroyed it very rarely regenerates.

Another prophylactic use of UV-C light is after the surgical removal of cancer cells or tumors. An uncertainty with respect to such surgical removal is that of insuring that all cancerous cells have been excised. Current procedures to ensure such full excision is the cauterization of the surgical site. This is, however, traumatic, with its own side effects and is prone to the possible missing of some cells or incomplete destruction of cancerous cells or possible precursor cells. UV-C trained on the surgical site provides a more effective destruction of any residual cancerous cells and a safe surface layer disruption of possible precursor cells to, at the very least, increase time of remission if not prevention of cancer recurrence.

### Deep Tissue Penetration Protocol

If required, a protocol to achieve deeper tissue penetration with superficial cancers such as described above, with emitted UV light, is by initially ablating a top or proximal layer of the mucosal tissue, typically with UV-C light, of about 20-40 μm deep. Additional ablative procedures, whether directly afterward or with additional treatment sessions, a next layer or successive additional layers of about 40 μm or even 100 μm of the impacted, or problematic, or cancerous cells or tissue are ablated. Treatment of cancer need not simply be the killing of cancerous cells. Instead, it is sometimes sufficient to wound cells, including cancerous cells, whereby a small inflammatory response is then triggered whereby macrophages or phagocytes come into play to consume and eliminate the cancer cells. This form of therapy is based on the fact that cancer cells do not have a repair mechanism, whereas healthy cells can repair themselves. The wounding of cells should not be of a nature which may result in cell mutation.

An advantage of the use of UV light is that the alternative, current, non-UV methods, use ionizing radiotherapy methods, such as irradiating with X-rays or gamma, etc., which can cause significant inflammatory response that is very traumatic to the adjacent healthy tissue or organ. This is in addition to the trauma of ablating or cauterizing methods used, such as with heat. In contrast, UV light triggers only a small or no inflammatory response with minimal trauma.

In a further embodiment of a transmission medium, a deeper tissue penetrating protocol, an optical-fiber with a UV transmissive needle-like distal end-treatment, or other end-treatment, is used to penetrate tissue and organs, transmitting UV DNA/RNA inactivating light directly into tissue layers, thereby destroying pathogenic activity, including cancer cells or tissue more directly and with minimized trauma to healthy tissue.

### Feedback Detection

It is very useful to be aware of pathogen kill times, and to effectively know achievement of pathogen kill times. In order to more effectively ensure confidence in effective pathogen eradication, pathogen killing UV-C, sufficient energy intensity should be maintained from the energy output, especially when transmitting the UV light at variable distances. It is initially understood that UV-C light is essentially not visible and tends to be dissipated or attenuated. Similarly, pathogens (especially in early stages) are not directly visible (though pathogen effect may be visible), nor are eradicated pathogens normally discernable.

A highly useful embodiment for use together with the UV light eradication of pathogens is one that identifies the pathogen activity, particularly the bacterial type, location and extent thereof, before, during, and after UV light application, i.e., feedback of eradication detection to the user or practitioner. Such feedback activity, before UV irradiation enables the more efficient and effective application of the UV light against the bacteria ensuring more effective eradication. Such feedback activity after a UV irradiation procedure serves as a helpful indicator of the effectiveness of the applied UV light eradication. It is understood that such feedback has use in other applications not related to the UV eradication such as in assessment of degree of pathogen infection and degree of amelioration of infection conditions.

In effect, with the collecting of eradication feedback during an exposure session, a “heartbeat” type profile, a real-time, connected-line plot, growing over time graph, of the bacteria is generated whereby a doctor, practitioner, or patient can see the initial activity level of the bacteria, prior to the UV being turned on. Declining levels of activity are then observed, culminating in the slowly flatlining-like portion of the graph as the pathogens are terminated by the UV, until substantially all of the bacteria is eliminated. If desired, even an enhanced form of time-lapse photography can be used to show a motion video of exactly where and how fast pathogen disinfection is proceeding. The enhancement is that rather than skipping all frames in between the sampled time-lapse frames, the time collapsing of a pathogen disinfection “movie” can via computer average the frames in between sampled frames, thus increasing the signal strength and signal to noise ratio, providing a better, higher dynamic range video, which still maintains the desired factor of viewing speedup.

The actual identity of the pathogen is less relevant since DNA/RNA, a universal characteristic of pathogens, is targeted. Instead, mainly the location and extent of bacteria is of interest. The field of Colorimetry, originally designed to facilitate the color matching of widely different additive and subtractive color reproduction devices, such as displays and printers, is useful providing the requisite information, though other methods are operable in accordance with the invention. Colorimetry is used, in one embodiment, to determine the extent and location of fluorescence, by comparing the perceptual color and brightness of each pixel using colorimetric methods, which numerically quantify the color perceived by a human eye, and can precisely compare that color to a similar view without the stimulating light. As an example, 405 nm visible light is used in one embodiment, with comparison of the color and brightness of that same pixel location after the stimulating light is turned on. Pixels which are of one of the known fluorescent colors, within a perceptual threshold, using the colorimetric differencing metric called “Delta-E”, used to compare two Lab values. The stimulating light is made to blink as it is On-Off modulated to directly indicate bacterial fluorescence with significant confidence. The extent of bacterial growth is indicated by the brightness of the fluorescence, and the combination of a precise color of the fluorescence and a correlation of the blinking nature to the blinking modulation signal combine so that, when both are detected or observed on epithelial tissue, a high confidence determination can be made of the existence of bacterial, or other pathogenic blinking.

It is a characteristic of bacteria that they fluoresce when exposed to light of specific wave lengths, with most bacteria fluorescing under light at 405 nm or smaller wavelength, usually generated by a specific wavelength LED. The wavelength used to cause the bacteria to fluoresce, is herein defined and referred to as the “stimulating wavelength”. Many different types of bacteria may be caused to fluoresce using the same or shorter wavelength (higher energy photons). The resulting wavelengths of the fluorescence of a different bacteria may be quite different, even if the same stimulating wavelength is used.

A direct, but complex and expensive, way to determine and categorize which type of bacteria is doing the fluorescing is to measure the wavelength of the emitted fluorescence-light, defined herein as the “fluorescence wavelength”. Identifying bacteria-types via its wavelength measurement utilizes special optical hardware, for example optical low-pass, high-pass, or band-pass filters and their combinations. Other hardware used to detect wavelength may be a spectrometer or a Bragg (adjustable) filter, including a “de-multiplexor configuration wherein successive sections of a special optical fiber with variable indices of refraction are used to filter out and branch off the multiple wavelengths sequentially.

The science of Colorimetry was originally designed to facilitate the color matching of widely different additive and subtractive color reproduction devices, such as displays and printers, are useful, though other color matching and discriminating methods are similarly operable, in accordance with the invention. Colorimetry encompasses: 1) Creation of the “Standard Observer” and quantification of his perceptions, 2) Creation of specific color spaces, specifically here “Lab” which facilitate quantifying color difference by providing color difference numbers very linearly proportional to the human perceptual difference, even over wide differences in color, and 3) Use of only number triplets, L, a, and b, to fully categorize color perception, rather than by manipulating wide spectral arrays of data covering the whole visible wavelength range. Using only three numbers to specify a color perception speeds calculations compared to operations on the wide visible spectrum. When dealing with whole raster frames of color pixels it is desirable to include fewer calculations per pixel to enable more video processing to be accomplished in software, with both flexibility and lower cost. (Note: an implementation using pre-computed lookup tables is especially fast at such color space conversions and differencing calculations).

The Colorimetric method of multiple fluorescence detection incorporates several techniques to ensure correctness and speed.
1) Pre-calculated Lab values for each and every fluorescence wavelength to be detected.
2) Time Synchronous CW (Continuous Wave, On/Off) modulation—The fluorescence stimulating light source is blinked at a specific rate. For a pixel to be counted as resulting from fluorescence, it, too, must blink in time with the stimulation light.
3) An optical notch filter may be used in the optical path of the detection camera to exclude blinking of direct stimulus light resulting from white or shiny surfaces.
4)The ‘a’ and ‘b’ components of the Lab value of a pixel's light should be within a small Delta-E difference from those of one of the pre-calculated list of Lab values for fluorescences.

The fluorescing provides a contrast with a patient's baseline pixel distinction and a color video clip (e.g., of one second or 100 frame grabs) is generated for analysis by fluorescing the tissue area and rapidly blinking the 405 nm or another fluorescing wavelength LED, to “call out” the pathogen. Alternative to colorimetry or in conjunction therewith, filters are used to separate color and intensity.

Feedback detection is useful in the effective treatment of bacterial infections such as throat infections or fungal infections, as well for life-threatening infections such as in a diabetic who may have foot ulcers that can lead to amputation if the pathogen isn't eliminated quickly from the wound.

With more specificity, when light of a certain wavelength shines on many live bacteria, they emit a very different, but specific, wavelength as if the bacteria were also a flashlight, but of a different wavelength. This is referred to as stimulated fluorescence. Like a flashlight, their emitted light stops in a fraction of a second when the flashlight is turned off. Thus, by blinking the light which shines on them, the light they emit also blinks along with it.

The amount of the live bacteria in the field of view can be determined by detecting a specific color of a fluorescence, and measuring how bright that emission is for that entire field of view. Different types of bacteria generally emit several wavelengths, many as a response to the same stimulating light, and a few bacteria may require a different wavelength for stimulation.

If there is also general illumination of the surface with the bacteria, detection of the bacteria is improved by ensuring that the bacteria's emitted light is being seen and not just the reflection of the general illumination, using an optical notch filter on the camera or detection device as described above.

There are two criteria to verify appropriate detection;
a) The first way is to verify that the detected light being looked at blinks in time with blinking of the stimulating light.
b) The second verification method is to verify that the wavelength being emitted is the same, or very close to, a known wavelength that bacteria emit when stimulated.

The method of paragraph a) is relatively easily employed since a stimulation light is blinked, which is usually with a highly controllable solid-state LED or laser.

The method of paragraph b), a wavelength detector is usually a general and expensive spectrometer, or, if only a few wavelengths need to be detected, a series of optical bandpass filters. Spectrometers and optical filters may be utilized with the former not having a wide field of view and the latter being limited as not being stackable together, since when one filter blocks all but one specific wavelength, all others, including the wavelengths of the other bacteria emissions are also blocked. Selection of one or the other or a combination is effective according to specific applications.

Fortunately, though each single specific wavelength, if coming from a monochromatic source like a laser, will be seen by human eyes as having its own distinct color, the reverse is not at all true. The specific perception of a color need not be produced by a specific wavelength or even a specific combination of wavelengths. Color film and all digital still and video cameras use combinations of light, from three rather non-overlapping spectrum sections of the whole visible spectrum, section approximately centered on Red, Green, and Blue, to effectively simulate the perception of all visible color. A virtual entity known as the Standard Observer has been defined. The observer's color perception is categorized by a different set of three numbers, one derivable from RGB space. A useful method for matching color perceptions from very different sources, such as lights (an additive space) and reflected of light from illuminated printed materials, is the “Lab” space, spoken of previously.

As a requisite for some procedures, preparation and removal of debris or other blocking agents, such as biofilm, plaque or mucus, etc., further helps the sterilization or inactivation processes go more smoothly. Limited penetration of UV light, generally is somewhat alleviated with a preparatory cleaning process with removal of debris which may block or hinder external applications such as endoscope reprocessing with sterilization of small channels such as the instrument channel, or with in-vivo medical ablation treatments, such as draining the mucus in a cyst, prior to deactivation of cyst tissue, or physically cleansing a wound prior to introducing the UV therapy for more effective treatment.

### Dentistry

In another embodiment, UV light in the disinfection wavelength range (in contrast to the upper, longer wavelength end UV commonly used to cure dental resins and implants), is used in the field of dentistry, whether human or animal, in the mouth, to help eliminate infection and rot on the surface of or inside an impacted tooth such as with cavities and to alleviate root canal conditions. Optical fibers are inserted into cracks and fissures of cavities to kill bacteria and prevent further progression of tooth decay. If the decay and bacteria are present on accessible surfaces. Collimated UV light is used to the same effect.

In a non-disinfecting aspect, the fibers used for UV light disinfection provides an imaging assist wherein a camera sensor image carrying fiber-bundle, used separately, or bundled together with a UV carrying optical-fiber, gains access into unreachable areas of organs, such as ducts, etc., or lymph nodes, to help view diagnostically and/or to administer UV therapeutics, with direct “line of sight”, without requiring the aid of more conventional viewing methods such as with ultrasound or x-rays.

This image carrying optical-fiber assembly with optionally UV-emitting output for disruption or inactivation of harmful or possibly harmful biological entities may further be used and inserted thru a fine aspiration needle, such as EUS or EBUS, to gain entry into organs or tissue, which otherwise is inaccessible from “direct” viewing. Effectively, becoming an endoscope within an endoscope, if inserted through a biopsy channel of an endoscope.

As used herein, “safe”, in addition to safety of humans, also includes safety to the device materials or instruments, ensuring that not too much energy output is directed at any one part of the device during sterilization causing device damage or degradation though, as it is known that excessive UV light (energy and/or duration) is known to degrade materials.

The above objects, features and advantages will become more evident from the following discussion as well as the drawings in which:

### SHORT DESCRIPTION OF THE DRAWINGS

FIG. 1 schematically depicts a side view of a pulsed 266 nm laser, as immovably anchored with fixed optical connection to a fiber optic cable with pathogen disruption output;

FIG. 1A schematically shows the 266 nm laser of FIG. 1 with components of a 808 nm laser diode converted to a 266 nm laser output with output power measurement;

FIGS. 1B-D show components and structure of a UV light emitting laser;

FIG. 2A illustrates a side emitting optical fiber modified to provide a uniform power output along its entire length;

FIG. 2B illustrates a stencil with fiber engraving lights for making the apertured side-emitting fiber of FIG. 2A;

FIGS. 3A, 3B and 3C schematically depict a cross section of an endoscope biopsy channel with a three UV LED structure in a staggered plane (FIG. 3A side view) and in a single plane (FIG. 3B end view) with a 360° radial UV light output; with FIG. 3C (side view outside of a channel) depicting a single LED with forward spread UV light output;

FIG. 4 schematically depicts a side cross section view of an optical fiber directing UV light at a surgical site from which a tumor has been removed and FIG. 4A depicts the application of a bladder with UV light being directed onto a site from which a cancer tumor was removed, for purposes of increasing length of remission;

FIG. 5A schematically depicts a view of an optical fiber directing UV light around the periphery of a tumor into healthy cells to provide an isolation of the tumor from the healthy cells, with FIG. 5B schematically depicting multiple depth peripheries of the application of the UV light around the tumor;

FIG. 5C shows an illustration of the tumor isolation of FIGS. 5A and 5B with a cross section side view of a colon showing varying stages of cancer development and an aspiration needle carrying a UV emitting fiber being used to form a dead cell and nutrient supply barrier between existing cancer cells and healthy cells with repeated insertions around the stage 2 tumor;

FIG. 6 depicts a multiple aspiration needle insertion of a cancer tissue coring operation which forms a widening lumen with repeated UV applications to kill surrounding cancer cells;

FIGS. 7A-C are cross section side views which sequentially schematically show the use of UV light in a raster procedure of removing cancer layers by disruption of RNA/DNA of cancer cells (FIG. 7A), then use of the UV light at high power to ablate and remove the dead cancer cells (FIG. 7B), and then using lower power UV light to again disrupt RNA/DNA of exposed cancer cells (FIG. 7C);

FIG. 8 illustrates a process for pre-cancerous near surface cyst removal by means of UV light use with cyst draining followed by internal cyst UV light deployment;

FIGS. 9A-B illustrate use of UV light deployment with an EUS needle carrier for pancreatic cyst drainage and residual cyst internal treatment with UV light;

FIG. 9C illustrates the UV light treatment on the interior surface of a stomach (stomach antrum) for treatment of intestinal metaplasia;

FIG. 10 shows a cancerous pancreas with duodenum access for an aspiration needle for delipidation and removal of pigments and subsequent insertion application of UV light;

FIG. 11A depicts the insertion of an aspiration needle through an endoscope into lungs for treatment by UV light of cancer in lymph nodes;

FIG. 12 shows an expanded view of a UV fiber as carried by the aspiration needle of FIGS. 10 and 11A;

FIG. 13 illustrates the collimating effect of specifically configured lenses in extending the output of UV light for a distance at relatively uniform and maintained power levels;

FIGS. 14A-C show various views of penlight shaped devices configured for emitting collimated UV-C light with exploded views and internal components;

FIGS. 14D and E respectively show a side cross section view of a human head and views of illustrating various specula for mouth insertion and aiming therein of UV light against the infections;

FIG. 15A shows an embodiment of a UV light emitting otoscope shaped device and its use in disinfecting a surgical site (FIG. 15B) which is a perspective view of a surgical operating site with the otoscope type instrument periodically bathing the surgical site with non-interfering disinfecting UV light from outside of the surgical instrument operation area and with a 405 nm LED light source for fluorescing bacteria;

FIGS. 16A and 16B are views of the display screen of the otoscope device of FIGS. 15A and 15B wherein the device emits light at a wavelength which fluoresces bacteria and the view originally depicting the non-fluoresced site (FIG. 16A) and depicts fluoresced bacteria at the viewing site for targeting (FIG. 16B);

FIG. 17 is a flow chart of feedback fluorescing and operation of the otoscope shaped device of FIG. 15A with fluorescing operation of surgical site 15B;

FIG. 18 is a ray tracing simulation of output of collimated UV, white and 405 nm fluorescing light emitted from the otoscope of FIG. 15A.

FIG. 19 shows the respective positioning of collimating lenses and UV, white and fluorescing light sources in the otoscope;

FIG. 20 depicts a nasendoscope currently used for nasal, throat and larynx treatment with inner ear reach, retrofitted with UV light emission for disinfection and bacteria infection treatment of such sites, and as being used for nasal disinfection;

FIG. 21 depicts a UV light carrying fiber being directed by a laparoscopic procedure to be positioned adjacent an infected medical implant pacemaker for the disinfection thereof by UV light in situ;

FIG. 21A is side section view of the medical implant pacemaker in a pocket between skin and muscle with insertion of the UV transmitting light fiber between the pacemaker and skin and pacemaker and muscle in the holding pocket;

FIG. 22 depicts a pre-UV light treatment elongated element with expansion element for disrupting bio-film in an endoscope biopsy channel and FIG. 22A shows it in position in expanded form for effecting the bio-film disruption;

FIG. 23 is a flow chart showing steps for removal of lipids and pigments from cancer tissue rendering it more transparent with less scattering of light from contained lipids and less light absorption by pigments allowing greater UV light penetration;

FIGS. 23A and 23B respectively show prior art tissue clearing progression over time of a mouse brain (FIG. 23A) and a tumor (FIG. 23B), to transparency;

FIGS. 24A-D show a full exterior of an endoscope being disinfected with timed endoscope insertion through a ring of UV light with minimized distance of UV surface applications, with FIG. 24A showing an endoscope contained in a protective glass and then rotated in a correspondingly sized cylinder with internal LEDs (FIG. 24B), and a vertically hanging endoscope with a small ring of LED lights being moved along the outer surface of the insertion tube (FIG. 24C) and a larger ring for the handle (FIG. 10D);

FIG. 25A shows a batch tray with an array of pipettes requiring disinfection and FIG. 25B depicts an array of pipettes being simultaneously disinfected with fiber insertion and UV treatment in batch trays on an assembly line with pipettes positioned for simultaneous disinfection with a movable matched array of UV transmitting optical fibers;

FIGS. 26A-B show a biopsy channel disinfection procedure with the device of the invention starting with power output determination (FIG. 26A), as an alternative to the integrated power measurement of FIG. 1A with FIG. 26A showing the free end of the fiber coupled to a UV light source inserted into an integrating sphere with power reading, with FIG. 26B showing the fiber inserted fully into an endoscope biopsy channel and being extracted by a retractor, with timing mechanism, on a linear track and moving at a calculated pre-determined rate;

FIG. 27 illustrates an embodiment of a sterilization box with collimated lens arrays for uniform and maintained power levels in disinfecting and sterilizing instruments (a scalpel is shown), such as used for surgery;

FIGS. 28A-C depict views of hand sanitization devices which emit UV-C light on proximately placed hands for the sanitization thereof;

FIG. 29A-C show dental application of UV light in a dentist office setting with UV treatment of cavities and gum disease (FIGS. 28A, B and C);

FIGS. 30A-B show the incorporation of UV light transmitting light element, as modified from the penlight configuration into surgical lights with camera elements.

### DETAILED DESCRIPTION

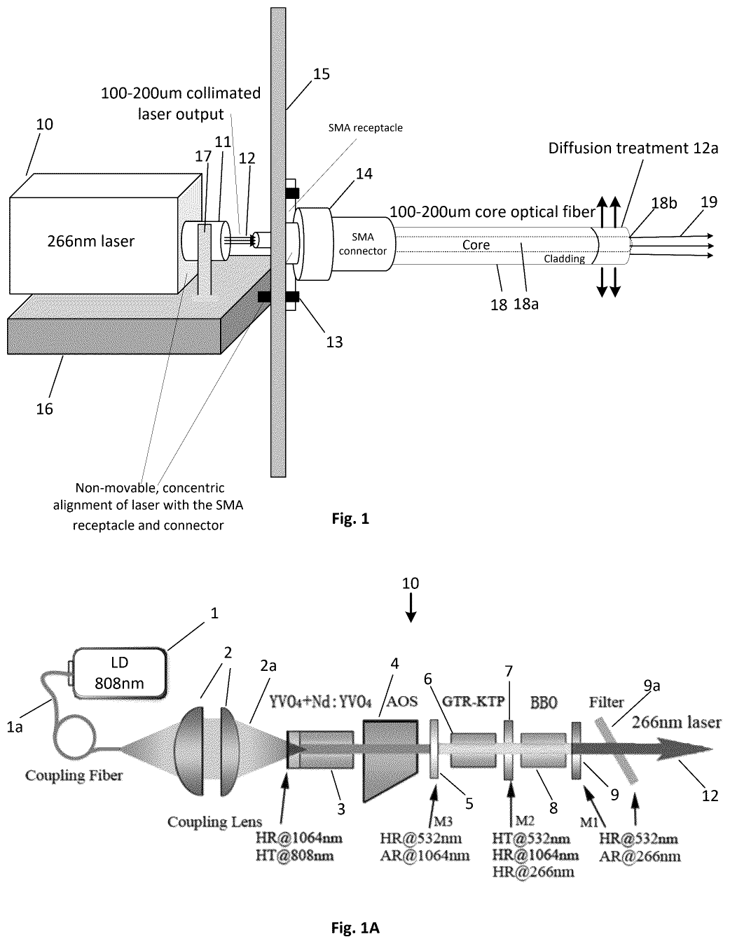

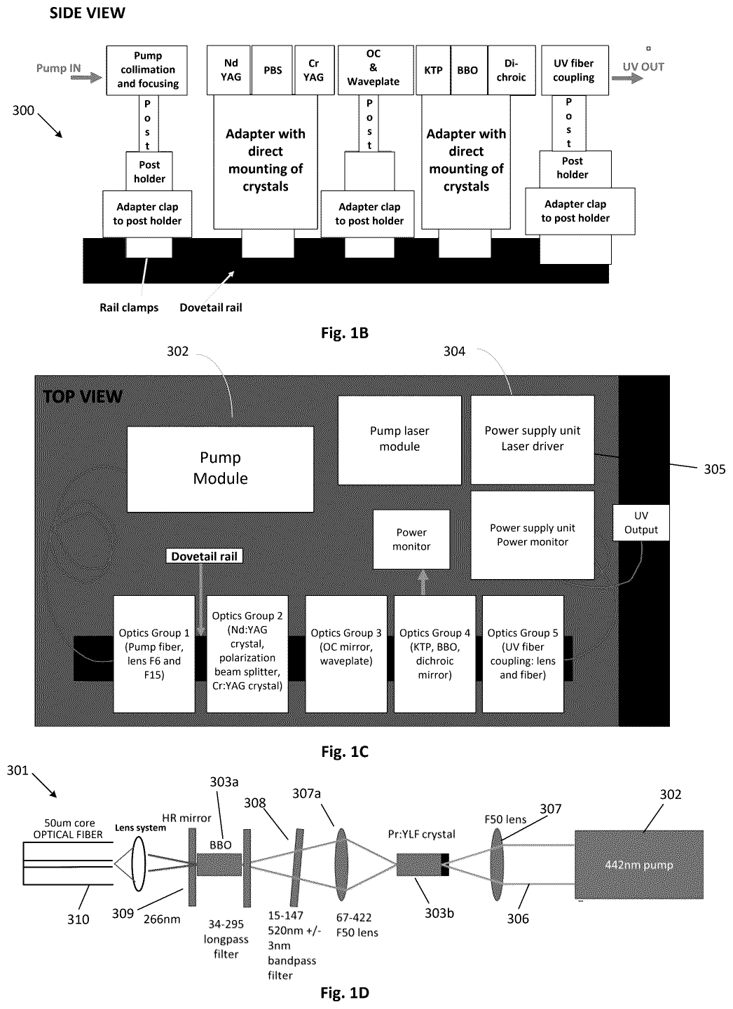

With reference to the drawings, FIG. 1 depicts a UV light emitting laser 10 with a 266 nm wave-length light (at or near the optimal DNA/RNA disruption level of 265 nm). The emitted UV light 12 is collimated and is in the form of a narrow beam of 100 to 200 microns which is optically aligned at brace 13 with an optical fiber 18 of the same or greater diameter through an appropriate acceptance angle (defined as an input angle which permits light to be accepted for output at the distal end of the fiber). With such small dimensions, the laser beam cannot be susceptible to slight movement which could cause the beam to be improperly angled and absorbed by the protective fiber optic shielding. Accordingly, the fiber 18 is locked into essentially non-movable position such as with glue or connectors to rigidly fixed brace 15, without play such as with the SMA connector 14 shown. The laser 10 is similarly locked into position on support 16 and with bracing element 17 locking output 11 in position. Though laser light 12 is collimated and non-diffused, its passage through the fiber 18 diffuses it slightly at the output 18b by about 0.5° in each direction. Further diffusion, as required for effective disinfection, is obtained by treating the distal or emitting end of the fiber 12a to effect light diffusion of desired spread. Alternatively, light may be emitted along substantially the entire length of the fiber.

FIG. 1A shows the components of laser 10, starting with laser diode 1 with 808 nm (which may also range up to 885 nm) with light output channeled via coupling fiber 1a into coupling lens 2 with the output light being focused at 2a into crystals 3 and 4 and conversion to 1064 nm and filter 5 into crystal 6 for second conversion to 532 nm. Thereafter, the light passes through crystal 6 and filter 7 and BBO crystal 8 and filter 9 to 266 nm laser output. Beam splitter 9a pulls some light out for measurement of power and the remaining light exits as laser light 12 at a 266 nm UV laser light output.

FIGS. 1B schematically shows a UV emitting laser configuration 300 with laser diode (200-280 nm) lens system to direct emitted UV light to a 50 μm core optical fiber with flexibility suitable for movable use within a body such as in an endoscope instrument channel (not shown).

Figures C and D show the typical components of a laser 301 with light generation 302 and transmission with wave-length modifying crystals 303a, 303b, power supply 304 and drivers 305. FIG. 1D illustratively shows a 442 nm pump laser with generated light 306 going through an F50 lens 307 to a PnYLF crystal 303b, an F50 lens 307a, a 520 nm bandpass filter 308, BBO long-pass filter 303a, an HR mirror 309 with a 266 nm (UV) light output directed by a lens system into a 50 μm core fiber 310.

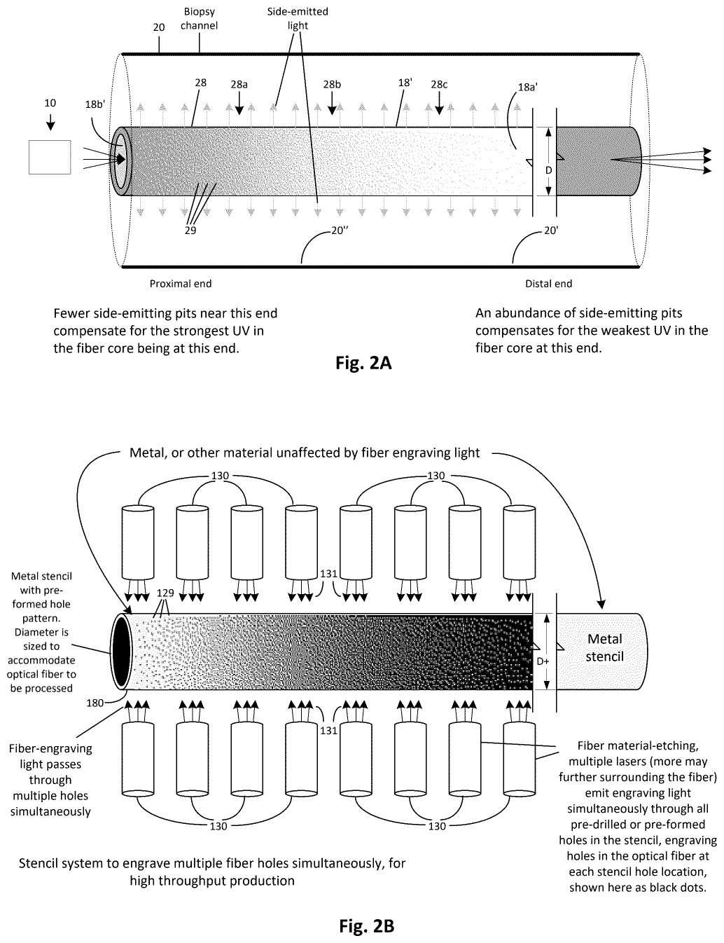

FIG. 2A shows a side-emitting fiber 18′ with cladding 28 which is perforated with microdot holes 29 to expose the fiber within the cladding for substantially full side emission of UV through the microdot holes with substantially uniform power output, as shown (or otherwise predictably controlled) output intensity. Though the microdot holes create small openings so that some of the light and output intensity can be emitted, the fiber remains sufficiently structurally sound to provide protection to the fragile fiber core. Effective pathogen eradication can be provided with such fiber structure with the use of a high-powered light source such as a laser or a high powered LED.

FIG. 2B shows cylindrical stencil 180, configured with calculated hole placement, and with an appearance corresponding to the fiber 18′ shown in FIG. 1A (with stencil holes 129 corresponding to the microdot holes 29 formed in the fiber 18′) is used to form the array of microdot holes 29 in a desired configuration on the fiber 18′. The cylinder 180 is of slightly larger diameter D+ than the diameter D of fiber 18′and sufficient for it to enclose the fiber 18′ therewithin during etching microdot hole formation. Stencil cylinder 180 is of a laser-etching impermeable material such as of metal (the laser light etchers 130 are configured to etch or perforate glass of the fiber buffer and cladding). Glass etching laser light 131 is trained on the stencil enclosed fiber to efficiently form the microdot holes 29 through stencil holes 129 in a desired configuration such as shown in FIG. 2A. Multiple laser etchers 130 surround the stencil to efficiently form the requisite microdot holes in the fiber 18′.

Simple side emitting of light (as in the prior art with LEDs with output in the range of 65 mW) or with uniform density of microdots (not shown) results in a structure which emits light from the side with drastic reduction in output intensity, with greater distance from the light source, often petering out to little or no pathogen eradication light emission at a distal end of an 8″ fiber. Density of graduated microdot holes distal end section 18a′ of fiber 18′ in section 28c emits at least milliwatts of UV light emanating originally from laser 10 whereby the distal end 18a′ effectively disinfects the distal end 20′ of biopsy channel 20 in minutes. The remaining sections 28a and 28b of fiber cladding 28 are configured along an appropriate calculated side emission power curve to be more opaque with fewer microdot holes to compensate for greater power emission of UV light from fiber section 18b′ closer to the UV laser light source 10 and a substantially uniform side output power along the length of the fiber.

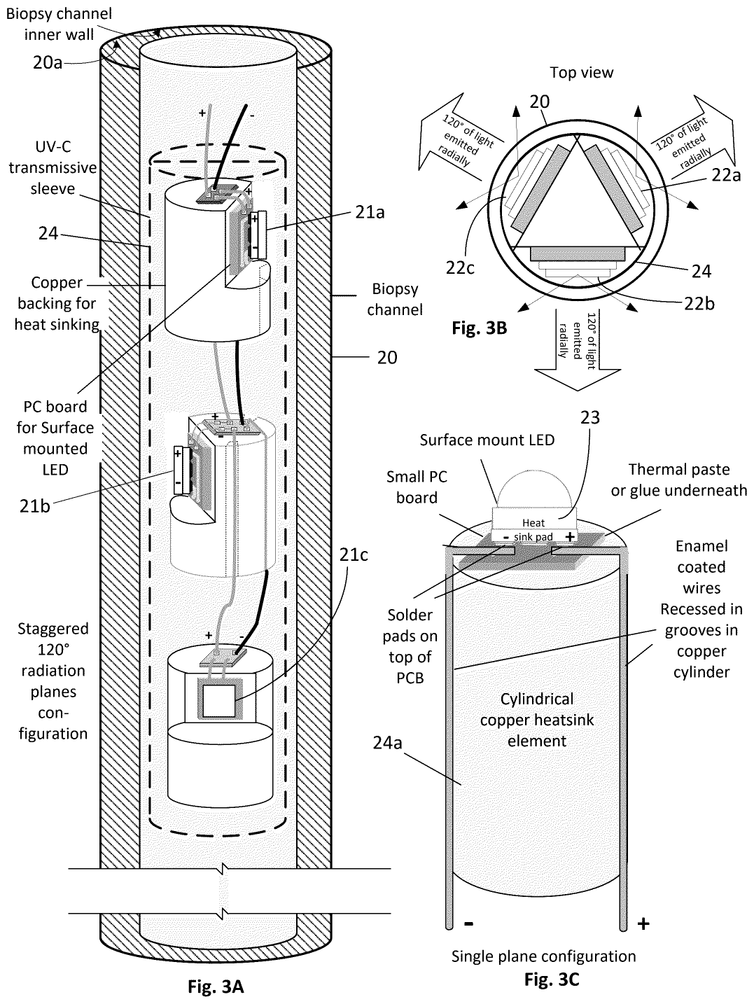

With an endoscope biopsy channel 20 or catheter of relatively large diameter, e.g., 4 mm or more, an offset linked vertical series of UV LEDs 21a, 21b and 21c contained within a transparent UV light transmissive sleeve 24 are inserted into an end of a biopsy channel 20 as shown in FIG. 3A. The LEDs each face the inner wall 20a of the biopsy channel and are offset from each other by 120° to ensure a 360° degree total UV light coverage of the inner wall 20a. During disinfection the LEDs are initially fully rotated to ensure full UV light coverage at the resting base of the LED stacked series prior to disinfection extraction.

In FIG. 3B an end view of the biopsy channel 20 shows an inserted alternative single planar version of the three LEDs 22a, 22b and 22c arranged in a single plane in a triangle configuration whereby the LEDs emit UV light respectively against the inner wall of the biopsy channel 20a in respective 120° angles.

FIG. 3C depicts a single LED 23 atop a cylindrical copper heat sink structure 24a, sized to be longitudinally placed in a biopsy channel with the LED providing a conical circular UV light output at an angle which impinges on the wall 20a of a biopsy channel into which it is inserted.

Though the LEDs, because of size constraints, are of lower power in sizes suitable to fit within the channel, full power without attenuation losses is directed against the channel walls. The LED arrangement is provided with power input and with a sufficiently rigid positioning rod, wire or cord, used for placement and controlled movement (not shown).

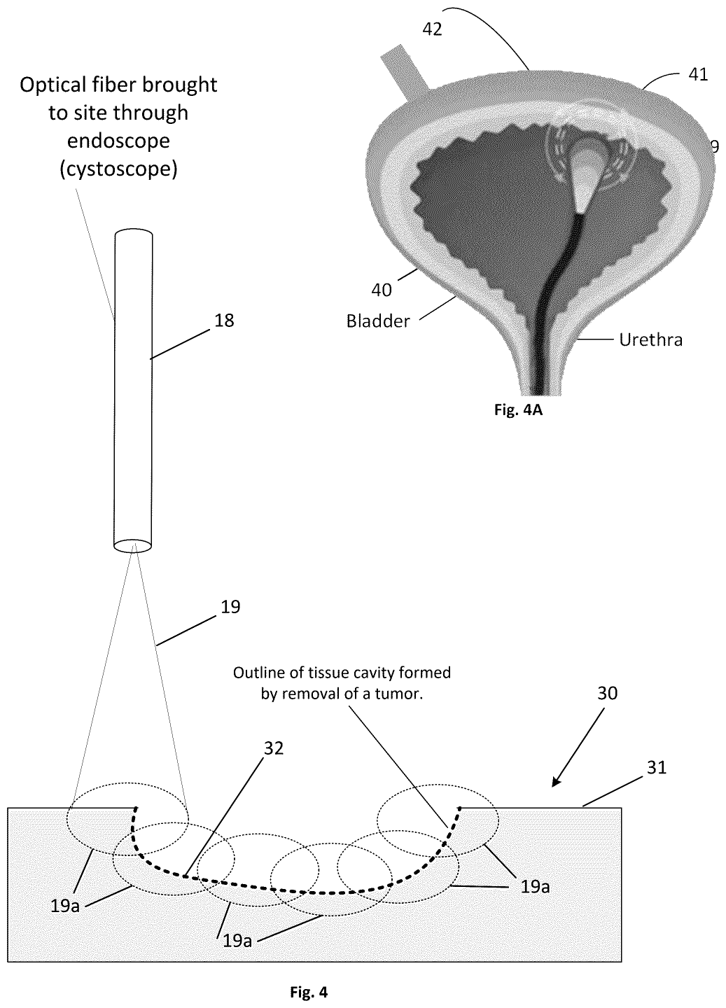

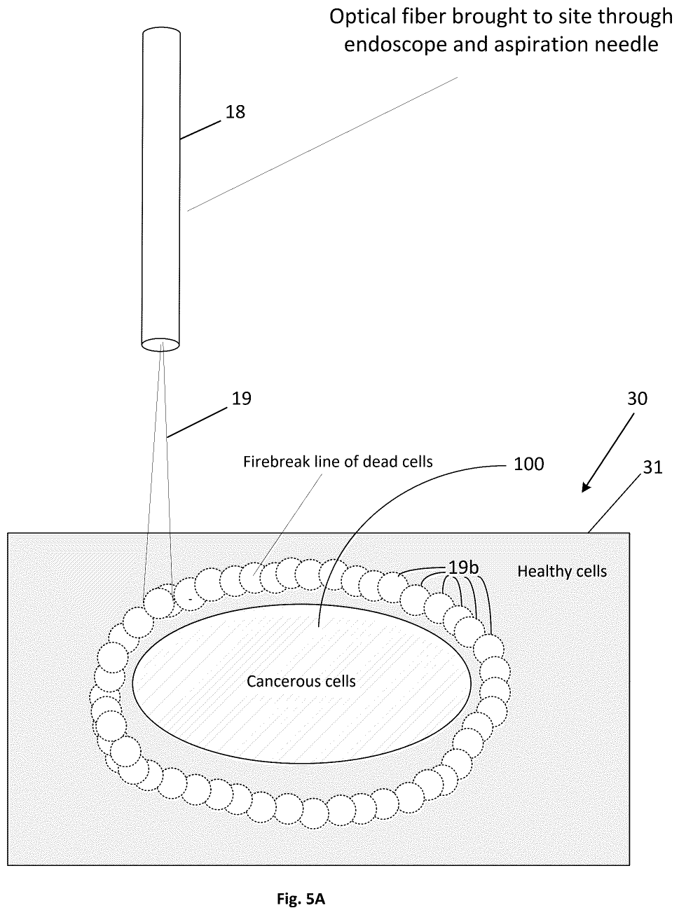

FIG. 4 schematically depicts a cancer tumor site 30 with a tumor having been surgically removed and the removal area 32 is shown in phantom in dashed lines. UV light 19 directed from a fiber 18, brought to the tumor removal site 30 by an endoscope (not shown), is prophylactically spread over at least the tumor removal and adjacent areas 32 and 31, as depicted by the circular areas 19a to retard return of aggressive cancers and increase duration of remission. This is akin to BCG treatment of bladder cancer sites with a surface treatment to prevent recurrence. The site treatment may be used in conjunction with or in place of current surface treatments.

FIG. 4A illustrates the schematic procedure in FIG. 4, wherein a bladder 40 has a removed tumor at site 42 on the inner wall surface 41 of the bladder. UV light 19 from fiber 18 inserted through urethra 43 is trained at least on the site 42 and surrounding tissue of wall surface 41 to maintain remission and prevent tumor regrowth.

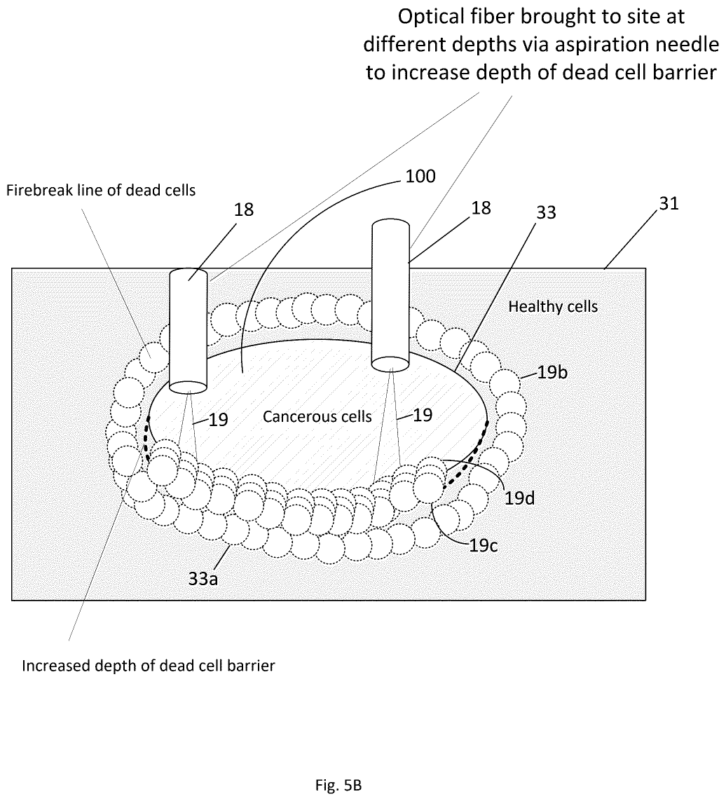

A cancer treatment protocol is schematically depicted in FIG. 5A wherein a cancer site 100 perimeter or interface 33 with healthy cells is detected and defined by biopsy procedures. UV light 19 from fiber 18 is directed at healthy cells 19b adjacent the interface perimeter, to form a separating line of dead cells 33a or “firebreak”, with disruption of cell nutrient pathways therein. As shown in FIG. 5B, with surface treatment being about 40 microns, greater depth for the “firebreak” or cancer expansion barrier is effected with an aspirating needle 18a extending contained UV light directing fiber 18 (carried to the site by an endoscope, not shown, through its biopsy channel as described in the parent application) which pierces into the area 31 at a lower depth of the interface to increase the depth of the “firebreak”, as shown, with increasing depth at 19c and 19d. Spreading of the cancer cells is contained thereby with a limitation on metastization. Phagocytes, which remove the dead cells provides a separation between cancer cells and healthy cells and blood vessels with nutrient supply is also disrupted in containing cancer growth and spread.

FIG. 5C is a sectioned depiction of a colon 105 showing various stages of cancer tumor growth ranging from non-colon-surface-penetrating tumor 99 to stage 1 (101) with slight surface-penetration to stage 2 (102) with surface layer penetration and stage 3 (103) full surface layer penetration and stage 4 (104) with full penetration and extension outside of the colon with spreading to other organs (metastization). Stage 2 cancer is illustratively shown (since the colon has a lumen and the cancers are surface type cancers surgery is best for removal) for treatment by the treatment protocol of FIGS. 5A and 5B with multiple needle penetrations with UV deployment from fiber 18 (19a) of the tissue around tumor 102 in order to build the “firebreak” of a dead cell barrier around the tumor to retard further spread of the cancer to surrounding colon surface tissue.

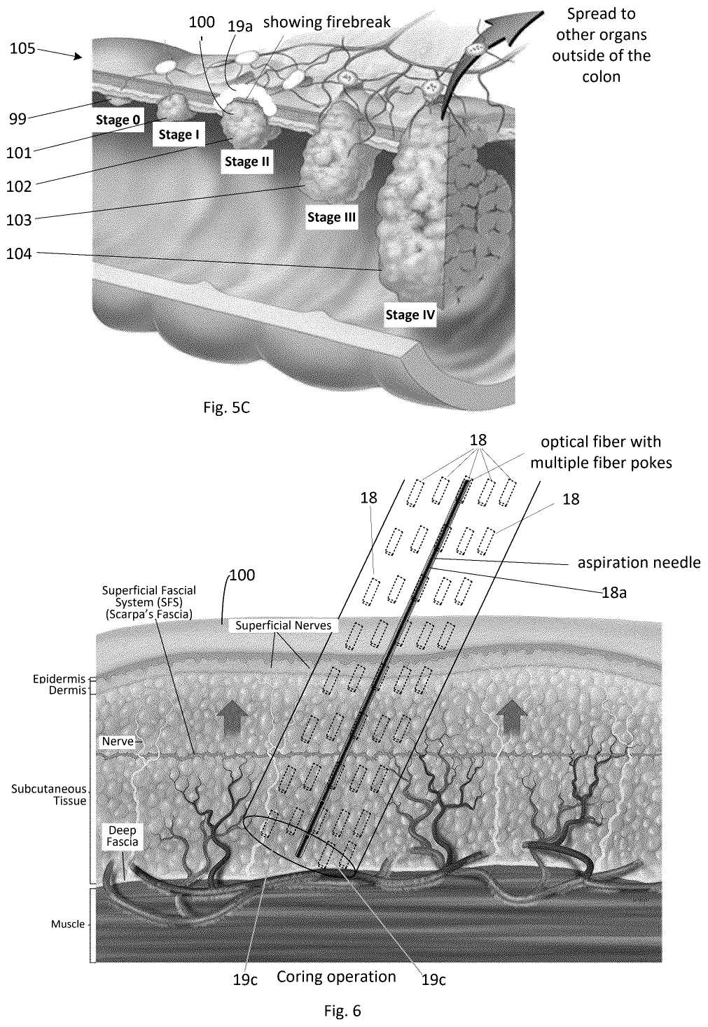

FIG. 6 illustrates another UV treatment protocol of a cancer site 100 with full needle penetration of the tumor 100 itself by aspiration needle 18a and then extending fiber 18 to laterally treat surrounding tissue. As shown in dashed lines, needle 18a with fiber 18 is withdrawn and inserted numerous times around the initial needle deployment to provide a coring treatment with expanding core areas of dead cancer tissue 19c. Ablative removal of cored tissue is effected through secondary needle deployment in the core area with applied suction or fluid washing (or phagocytes allowed, in time, to remove the dead cells) to widen a small lumen within the tumor to allow for increasing widening of the UV treatment and successive disruption and formation of dead cell layers for continued removal.

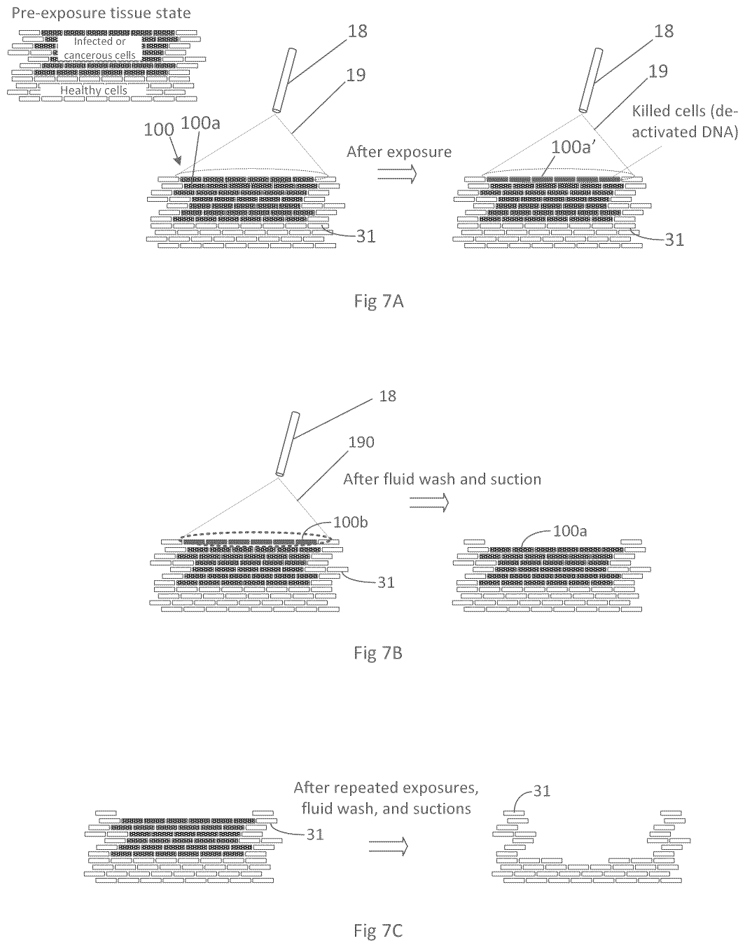

FIGS. 7A through 7C illustrate such widening cancer cell removal with fiber 18 shown as directing UV light on (or in) cancer tissue of cancer 100 which is located in healthy cells 31 (the cells are schematically depicted as building blocks of light and dark bricks of healthy cells 31 and cancer cells 101a respectively). Raster deployment of UV light 19 at a disruption power level kills the cancer cells in the layers exposed to the UV light (FIG. 7A) and the dead cells are then treated in a raster deployment by ablation power levels of UV light 190 with dead cells removal by a fluid wash and/or suction as shown in FIG. 7B. The power levels of UV light disruption (generally in the double digit milliwatt range) are sufficient to kill cancer cells which cannot self heal as compared to healthy cells which can. Dead cells have lessened intercellular adhesion and are more easily ablatively removed. The disruption/ablation raster procedure is repeated (preferably automatically or robotically to successively kill and ablatively remove cancer tissue cells 100a as shown in FIG. 7C to lessen tumor size or to remove it.

Non traumatic removal of cancer cells shown in the sequential depiction of FIGS. 7A-C in which a surface layer of cancer cells is treated with UV light to the point of DNA/RNA disruption and eradication (FIG. 7A). Thereafter, UV power level is raised to minimal ablative power and trained on the dead surface cancer cells to effectively sweep them away (FIG. 7B). The exposed new surface of the cancer cells is then again treated with UV light with a raster-like repetition of disruption and ablation. Depending on duration of each of the cycles and assuming a ten second period of the cycles and a 40 micron removal for each cycle, removal of a 1 cm thick cancer layer requires about 2500 seconds or about 42 minutes of total treatment per cancer site. The disruption is on a molecular level and not physically discernable and the ablative cycles are at minimal effective power to remove dead cells (less power than required to ablate live cells) with minimal trauma, if any, and certainly much less than standard radiation therapy by orders of magnitude. The endoscope carrying the optical fiber used for the disruption/ablation procedure has water, air and suction channels which are used during or after ablation procedures to facilitate cleaning and removal of dead cells with exposure of additional cell surfaces. Bio-feedback such as cancer cell luminescence provides visible indications of the effect of the cancer cell removal.

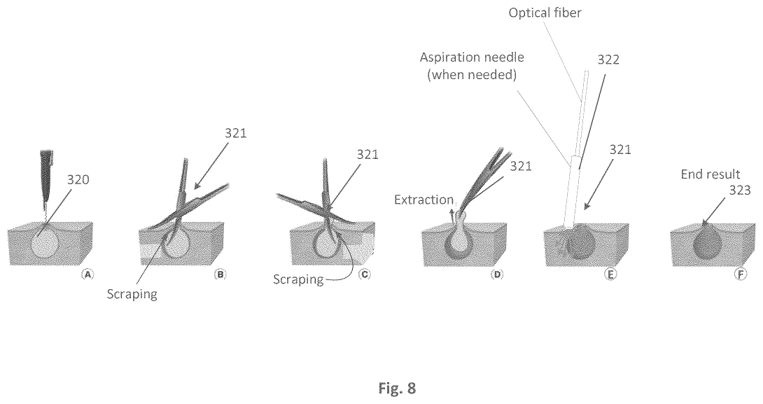

FIG. 8 schematically illustrates cyst 320 removal by surgical excision 321 (FIG. 8, in steps A-E) and subsequent UV light treatment (step F) via an optical fiber carrying UV light to the bared surface of the cyst to prevent further growth such as cancer cells 323.

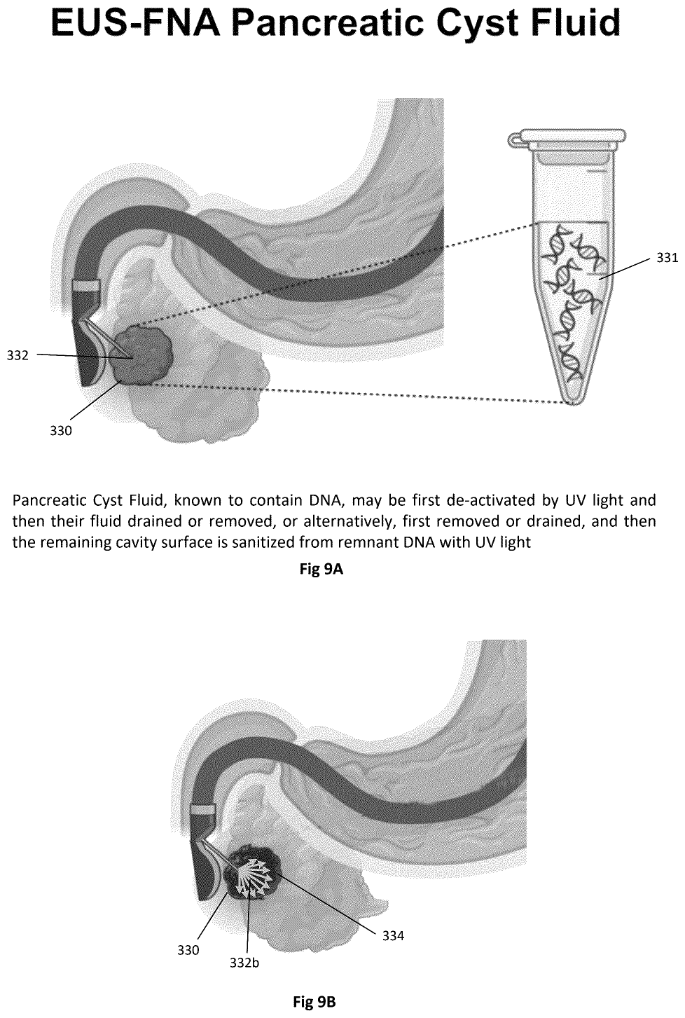

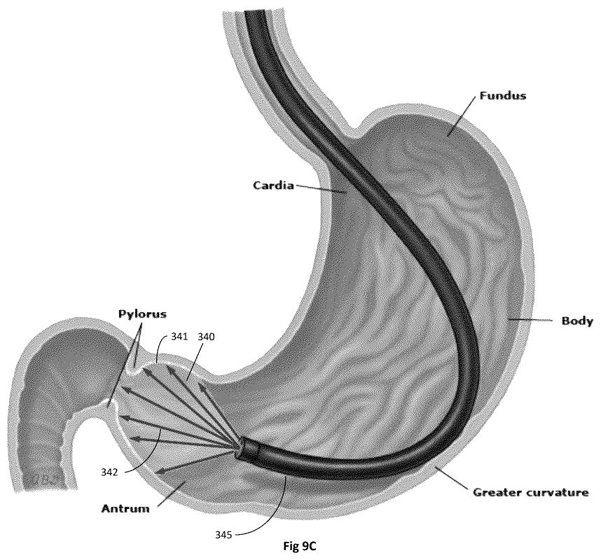

FIGS. 9A and 9B show a specific prophylactic deactivation of pancreatic cysts 330 (the precursor of the vast number of pancreatic cancer occurrences). As shown in FIG. 9A, pancreatic cyst fluid 331 (having DNA) is deactivated by UV light 332 and the fluid 331 drained. Alternatively, the fluid 331 is initially removed, and the residual cavity surface 334 is treated with UV light 332b (FIG. 9B) to prevent pancreatic cancer generation therefrom.

FIG. 9C shows the application of UV light 342 deactivation treatment to the stomach antrum 340 epithelial cells 341 which are prone to intestinal metaplasia by becoming intestinal cells. Since such cells are not discernable, they are nearly impossible to be surgically removed. Instead, UV light 342 is delivered with an endoscope 345 to reach the inner stomach lining, in the region susceptible to intestinal metaplasia and the entire surface is “photonically ablated” from the intestinal cells in thin layers, thereby allowing fresh epithelial cells to grow and replace them.

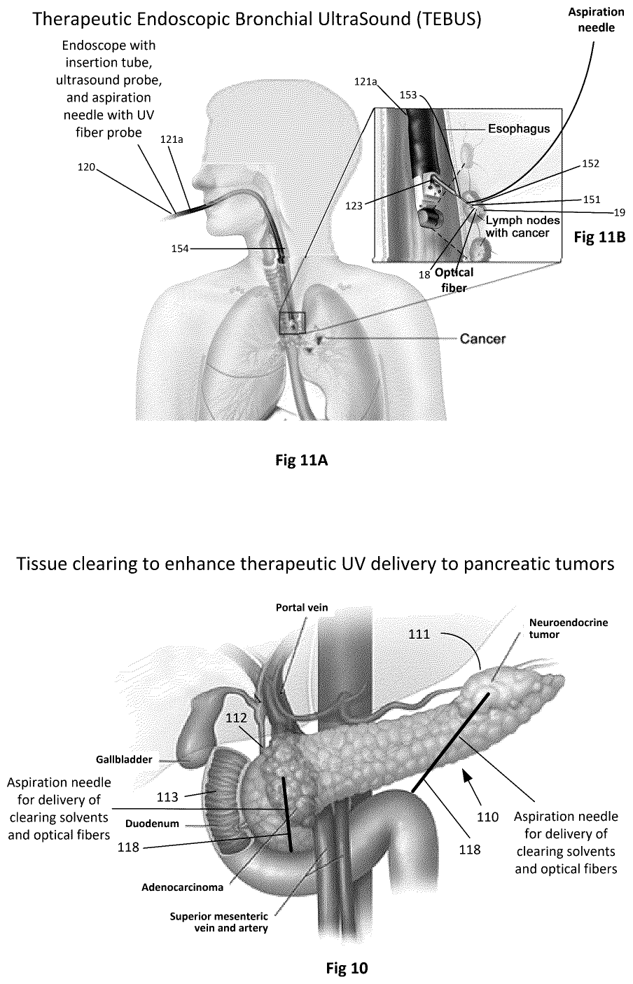

As illustrated in FIG. 10 UV light treatment of pancreatic cancer shown on pancreas 110 as tumors 111 and 112 is facilitated with initial tissue clearing of the tumors with clearing chemicals being inserted into the tumors by aspiration needles 118 with access to the tumors through the duodenum 113 adjacent the pancreas. The tumors are rendered more transparent to light whereby UV light subsequently provided to the tumors with the aspiration needles has an effective increase of penetration from about 40 microns to at least one to several millimeters whereby the UV light provides an effective non traumatic tumor killing expedient not previously available. This procedure is effective as against any cancer since they all contain DNA/RNA which is disrupted by UV light. While a needle or similar device is used for delivery of chemicals for tissue clearing, the fiber itself, as well as the needle, may be used for penetration into the cancer tissues for direct delivery of the cancer disrupting UV light.

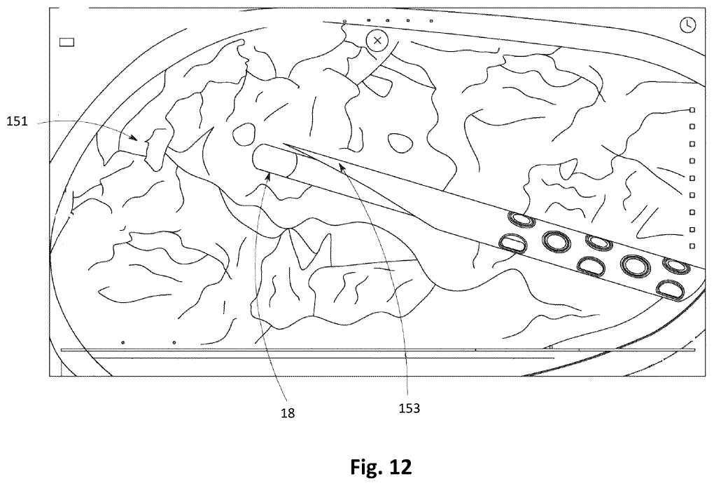

FIGS. 11A and 11B illustrate the use of an endoscope 120 to carry an aspiration needle 153 to a cancer site tumor 151 on lymph node 152 with the needle 153 (used in the prior art to carry ultrasound instruments for mapping and therapeutic treatment of tumors EBUS and TEBUS) to carry a UV emitting fiber to the tumor and also solvents for rendering the tumor transparent. As shown, the endoscope 120 is inserted into the esophagus 154 of the patient and the insertion tube 121a of the endoscope is brought into a position wherein the needle 153 is carried into proximity to the cancer site 151. As shown in the expanded view of FIG. 11B, elevator 123 of the biopsy channel within insertion tube 121a controls placement of the needle 153 relative to the tumor 151. Control of the needle 153 is independent of control of the fiber 18 contained therein (shown in FIG. 12) whereby the needle 153 with contained fiber 18 is inserted into the tumor 151 and the needle 153 is retracted to leave the UV emitting distal end of fiber 18 imbedded within the tumor 151. UV light transmitted through the fiber 18 as light 19 effective kills the pathogenic (as defined) cancer cells from within the tumor, without significant effect on healthy cells.

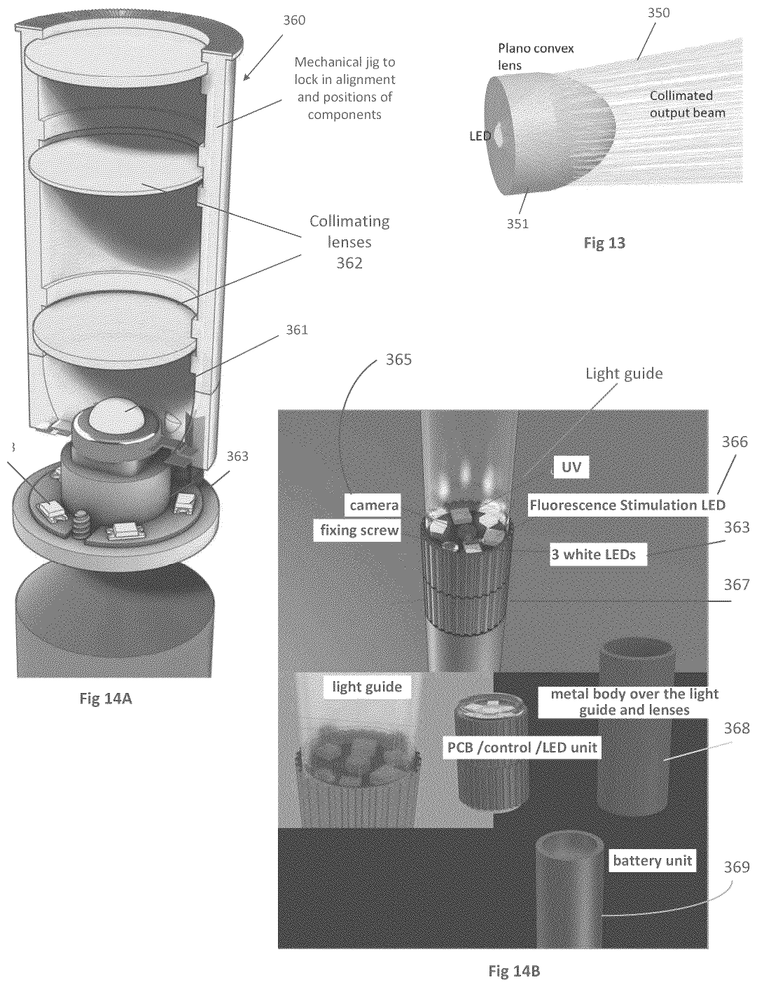

FIG. 13 schematically illustrates collimated output beam 350 from a single LED light source 351. Multiple arrays of LEDs provide increased output power and/or increased surface area for disinfection. A single collimating lens 350 in FIG. 13 and an array of collimating lenses in maintain output beams with controlled substantially uniform diameters over a pre-determined collimating distance. This output is suitable for maintaining substantially full effective DNA/RNA disrupting UV power in relatively short distance applications.

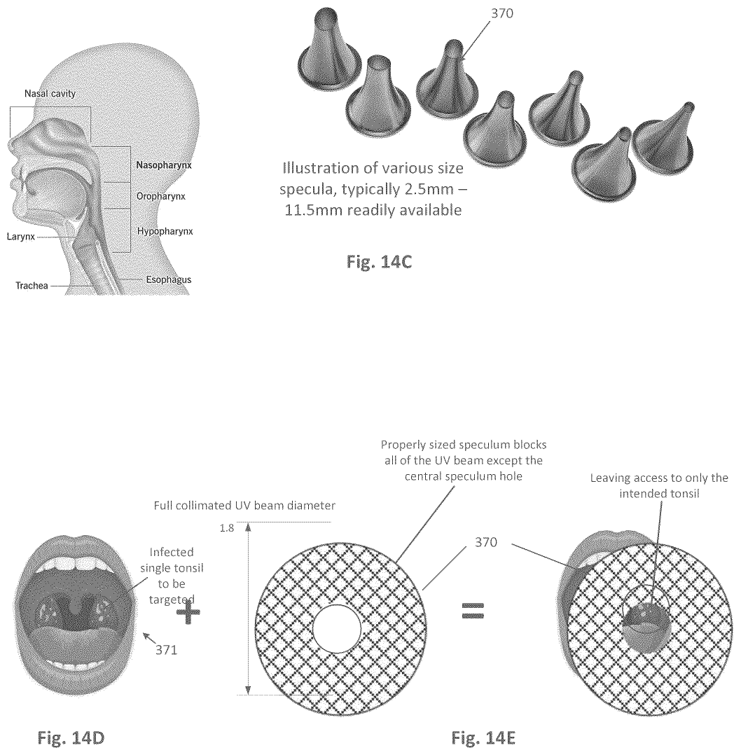

FIG. 14A depicts one embodiment of a penlight type device 360 which is configured to emit collimated UV light. The small diameter of the penlight device 360 is between about 15 to 20 mm to permit its ready use in a child's mouth, as will be described. FIG. 14B is a partial cross sectioned open views of the optical section of the penlight including LED emitting element (UV 361 and illumination 363) lights with collimating lenses 362 within placement locking enclosure having a protecting planar transparent closure.

As shown in FIG. 14B, a camera 365 is included to detect reaction from a fluorescence stimulation LED 366 which causes bacteria to fluoresce at specific wave lengths. This enables detection of effectiveness of the UV in deactivation of bacteria and/or viruses and for proper aiming of the penlight device at the pathogens. A ribbed control element 367 is used to control the light emission and an optional light guide prevents stray UV light from exiting away from a designated path. A metal container body 368 is used to avoid UV degradation of plastic or polymeric elements. A rechargeable battery pack 369 is encased in a distal area of the penlight structure, with the battery providing at least a half hour of typical UV light use and with the battery pack being capable of being swappable with a replacement one and for separate charging. Various size specula 370 are shown in FIG. 14C for detachable use with the penlight device.

Utilization of the penlight device 360 is illustrated in FIG. 14C as a side cross sectional view of bacterial and viral infection sites of nasal cavity mouth and throat. FIGS. 14D and 14E schematically show areas 371 where UV light is directed for maximum pathogen deactivation by means of the appropriate speculum.

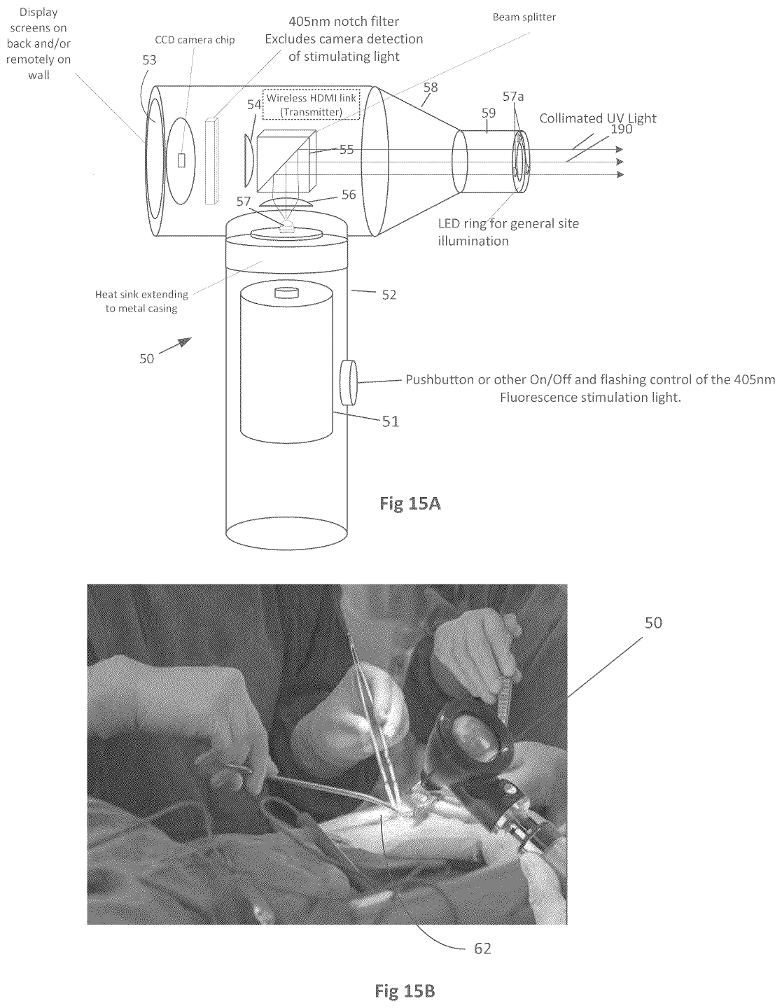

FIG. 15A shows a cross section view of an otoscope type device 50 generally used for the application of light into externally exposed orifices such as respiratory, alimentary and excretory orifices and viewing of these sites. The device is modified for the effective transmission of UV light and bacteria fluorescing light to an area infected with bacteria or other pathogens. The device 50 is provided with self-contained power supply 51, in handle housing 52 (a power line for additional power may also be attached to the handle), which powers UV pathogen-disrupting emitted light from UV LED 57. The UV light is transmitted and directed through a transmission medium of a collimating lens 56 and beam splitter 55 to speculum 58, which is aimed at the bacteria or other pathogen infected site.

The otoscope 50 configuration includes the ability to snap on removable specula-type adapters 58 that may include further optics such as lenses, fibers or light pipes, to gain further access into inaccessible or difficult to reach areas, such as the Eustachian tube, trachea, sinuses, surgical openings and wounds, including access openings for disinfection of implanted devices.

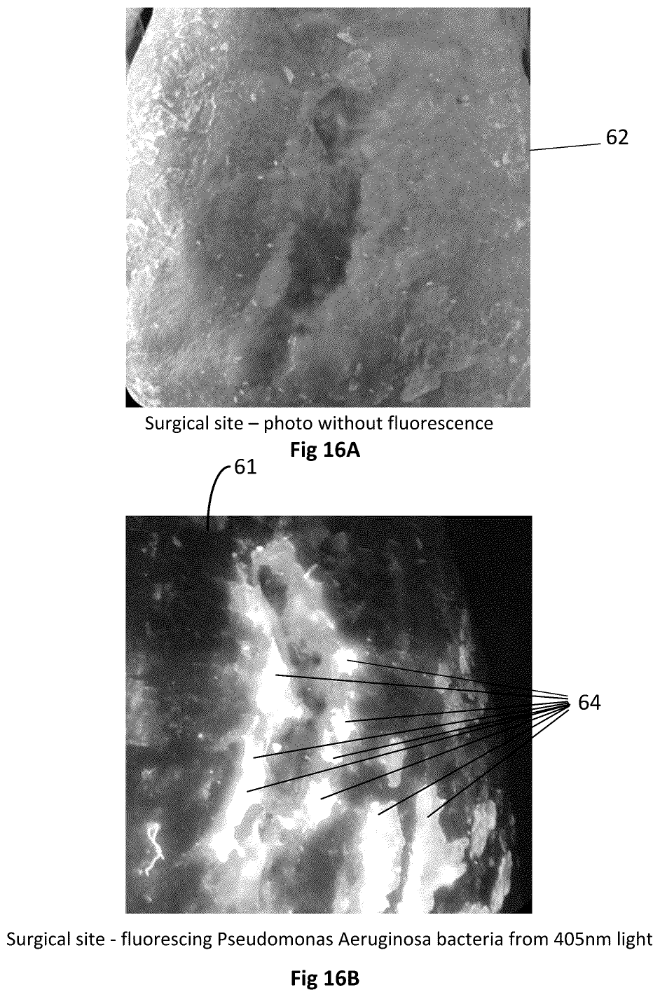

Reduced conical section 59 has external distal white LEDs 59a for illumination of the target site. CCD camera chip 53a receives the image of the aimed area and transmits it for viewing on screen 53. UV light 190 emitted from conical section 59 bathes the viewing site with UV disrupting light for killing pathogens in the viewed area. As shown in FIGS. 16A and 16B, light of a wavelength such as 405 nm fluoresces bacteria 64 of surgical site 62 and the otoscope device includes an LED 57a which emits such light on demand whereby the user can see the fluoresced bacteria 64 on viewing screen 61 and can move the aim of the otoscope device 50 to direct such fluorescing light and UV light for facilitated pathogen eradication at bacteria infected sites 64. Alternatively, the source of the fluorescing light which may comprise several sources (with differing waves lengths which fluoresce different bacteria) may be incorporated in the ring 59a together with the white light illumination LEDs

The target sites include sore throats in mouths or surgical operation sites 62 as shown in FIG. 16D. Trained collimated UV light from devices such as the otoscope like device 50, when used before, during and after surgery markedly reduces even the most disinfectant resistant bacteria from the site with removal of MRSA and reduction, if not elimination, of SSI. Though shown as handheld in FIG. 15B, the device can be supported on a movement-controlled stand (out of the way of the surgical procedure) and may even be automatically guided by feedback supplied by fluorescing of bacteria for the application thereto of UV light 190. Though the UV light is shown as being offset from the generating LED by 90° it can be more directly applied with rearrangement of the light source in the device to directly output the UV light from a transmitting lens directly through output section 59.

The UV light emitting otoscope shown in FIG. 15A, is similar in concept to the penlight configuration of FIGS. 14A-D but with a larger battery in a handle and an otoscope configuration. The UV emitting LED is in the handle and directs the UV light to a beam splitter which directs it to a collimating lens for transmission out of the speculum end substantially uniform collimated light with maintained UV disinfecting power for a distance of about six to eight inches, sufficient for entry and disinfection of the human orifices such as mouth nose and anus. The otoscope is provided with a display screen and ccd camera for direct examination of bacteria fluoresced areas stimulated to be recognizably visible by a 405 nm stimulating LED. The camera display is viewable on a separate enlarged screen via an HDMI which wirelessly transmits images and videos to a screen-linked receiver. The otoscope is usable, as shown in FIG. 15B in disinfecting and viewing bacterial disinfection of a surgical site. FIG. 15B and illustrates the viewed image of the state of bacterial disinfection in real time during surgical procedures.

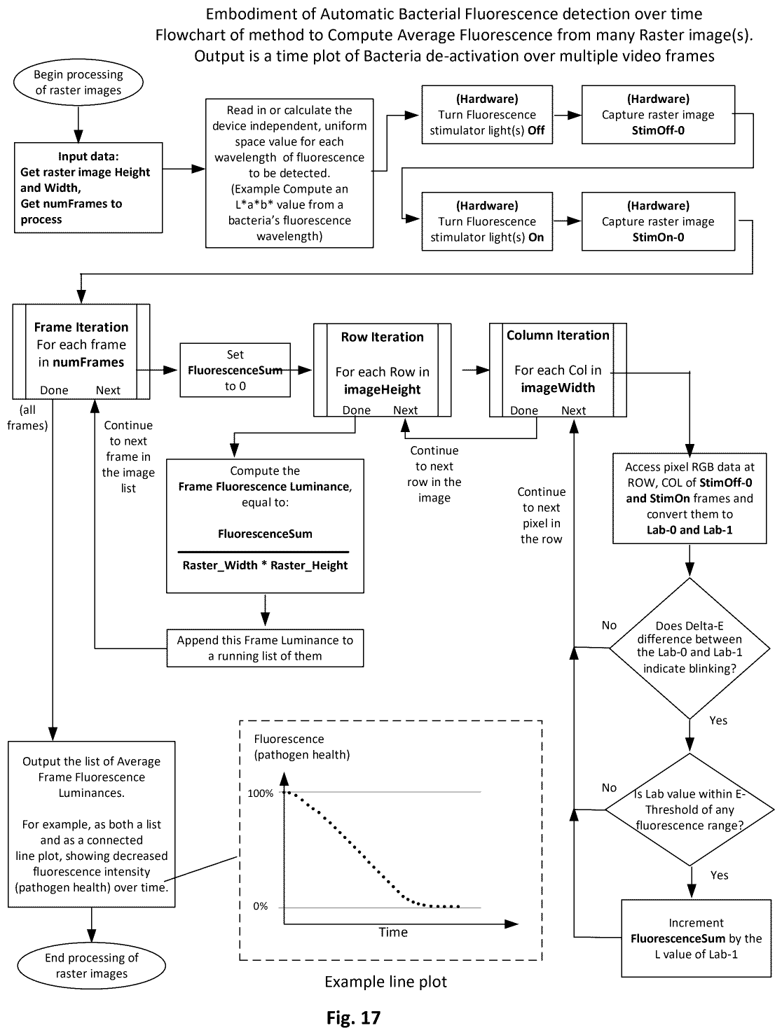

FIG. 17 is a flow chart showing a process for bacterial fluorescence detection such as for use with the otoscope and during the surgical procedure depicted in FIG. 15B. The process starts with a raster imaging of the site being examined after a calculated fluorescence illumination of the site. With calculations of the raster captured fluoresced images of varying colors and intensities (relative to various types of bacteria and their respective fluorescing reactions), pathogenic activity of bacteria is determinable. Both the initial detection of pathogenic sites (with intensity being indicative of degree of bacteria infection) and decrease in intensity being indicative of UV light disinfection effect in real time.

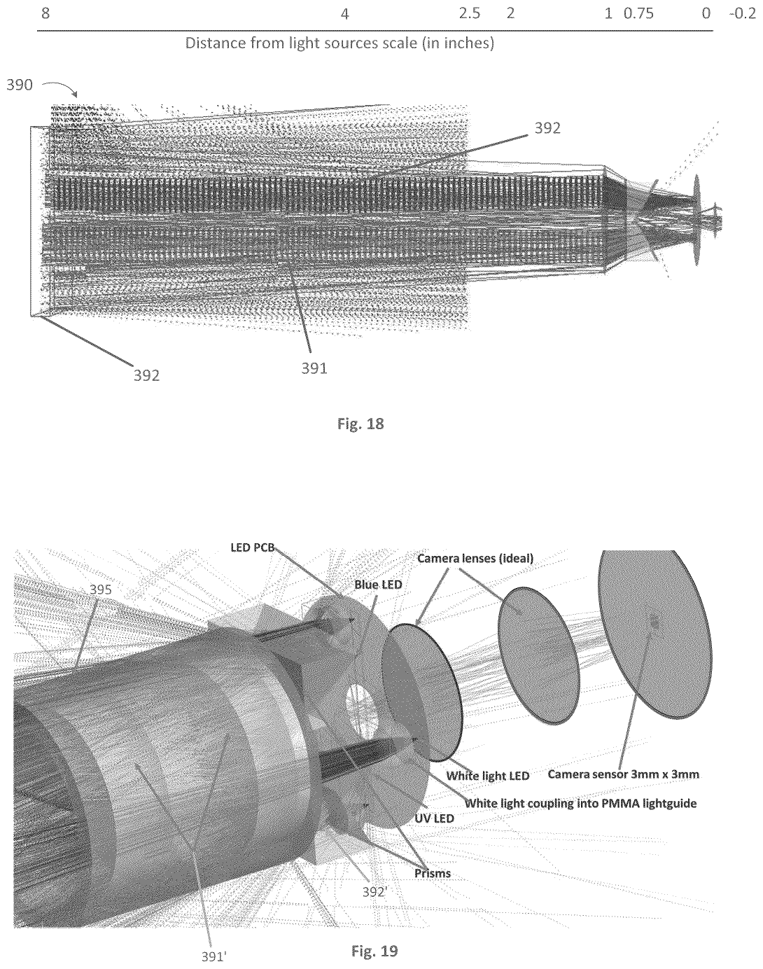

FIG. 18 is a ray tracing simulation 390 of output of collimated UV 391, white 392 and 405 nm fluorescing 393 light emitted from the otoscope of FIG. 15A. FIG. 19 shows the respective positioning of collimating lenses and UV 391′, white 392′ and fluorescing 393′ light sources in the otoscope with an expanded view of the optical system of the otoscope of with white light illuminating lightguide 395, UV, white and blue LEDs (indicator of UV light generation being active) UV and blue light collimating lenses, camera lenses and camera sensor.

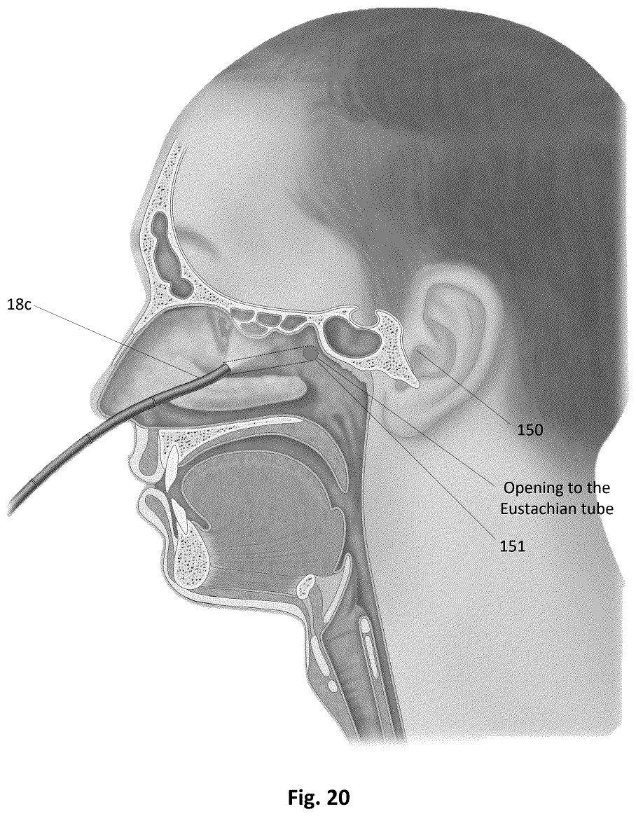

FIG. 20 depicts a currently utilized nasendoscope configuration 50′ fitted with a UV carrying fiber 18c (and self-contained UV light source and power supply—not shown) for effecting bacteria disinfection in areas requiring fiber extensions for being reached such as inner ear 150 with ear infection, through Eustachian tube 151.

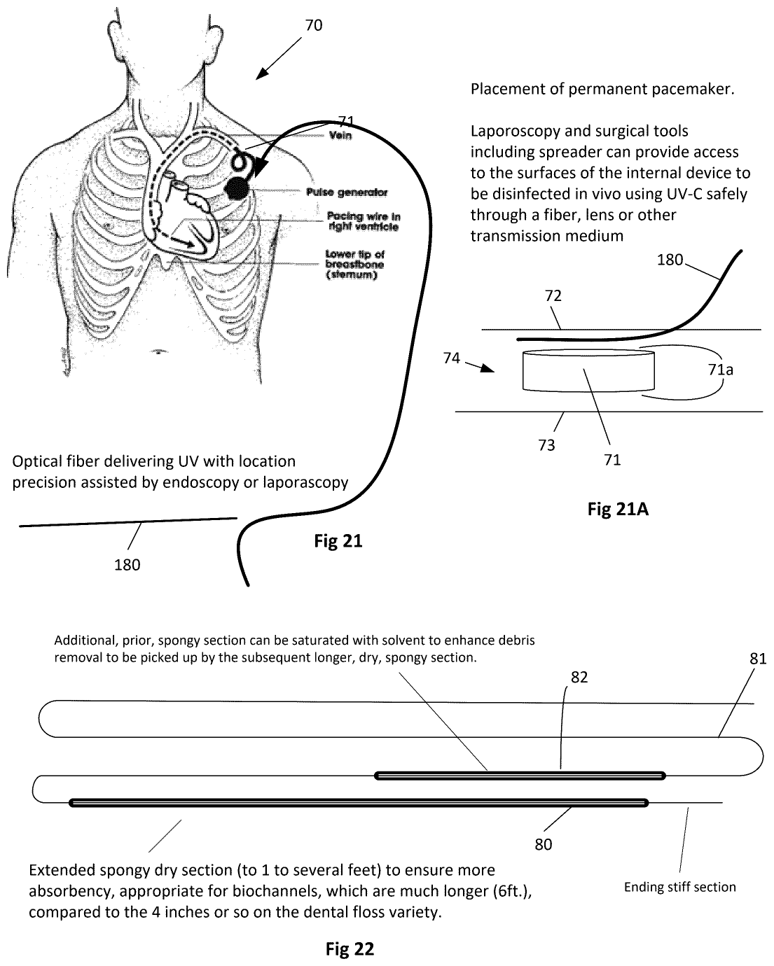

FIG. 21 shows a cross section view of an implanted pacemaker 71 in a human with a UV optical fiber 180 used to disinfect the pacemaker in situ. FIG. 21A shows the disinfection procedure in a cross-section view of the implanted pacemaker into pocket 74 between outer skin 72 and muscle layer 73. UV carrying fiber 180 is inserted into a skin incision adjacent the pacemaker and then moved across the surfaces 71a of the pacemaker to effect disinfection in situ without the need for surgical removal and attendant complications. To facilitate disinfection and since the fiber is inserted laparoscopically rather than through an endoscope, the fiber may be provided with a broadened UV light dispersing end which can also serve as a wedge to separate the surrounding skin from the pacemaker surfaces. Other implanted devices may be similarly disinfected in situ.

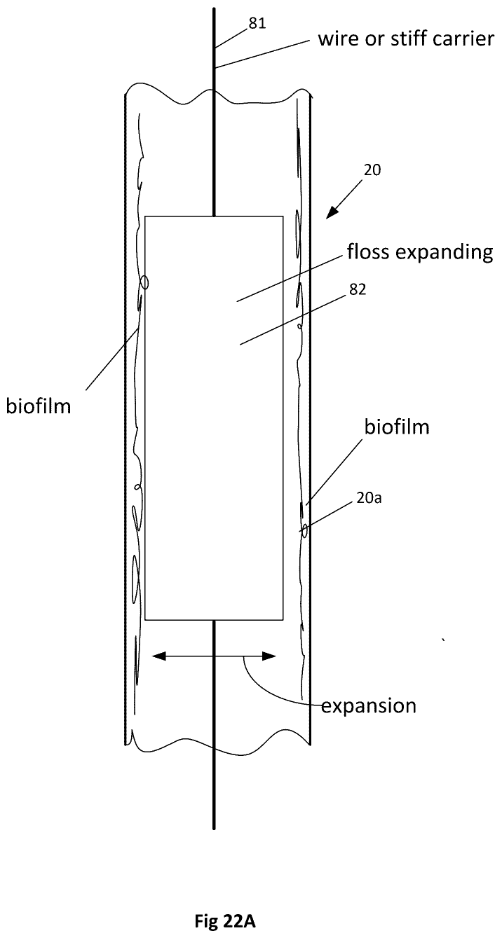

FIGS. 22 and 22A illustrate an additional embodiment used with endoscope biopsy channels, particularly with endoscopes which have not been immediately sterilized and a bio film has started to form over pathogens contained within the biopsy channel. This bio-film may retard the effect of UV light on the pathogens by limiting penetration. A combination wire with integrated absorbent material 80 serves to disrupt the bio-film and any UV light blocking characteristics. The relatively stiff wire element 81, with spongy absorbent section 82 (similar to a dental floss type configuration) of the combination 80 is inserted into the biopsy channel 20 prior to insertion of a UV light emitting fiber, as shown in FIG. 22A. The absorbent section 82 expands radially in the direction of the arrows, with full engagement with the channel walls 20a and biofilm 83. The absorbent section material 82 disrupts any biofilm by absorption and physical engagement particularly when the absorbent section 82 is moved by retraction of the wire support 81 and removal of the combination from the biopsy channel.

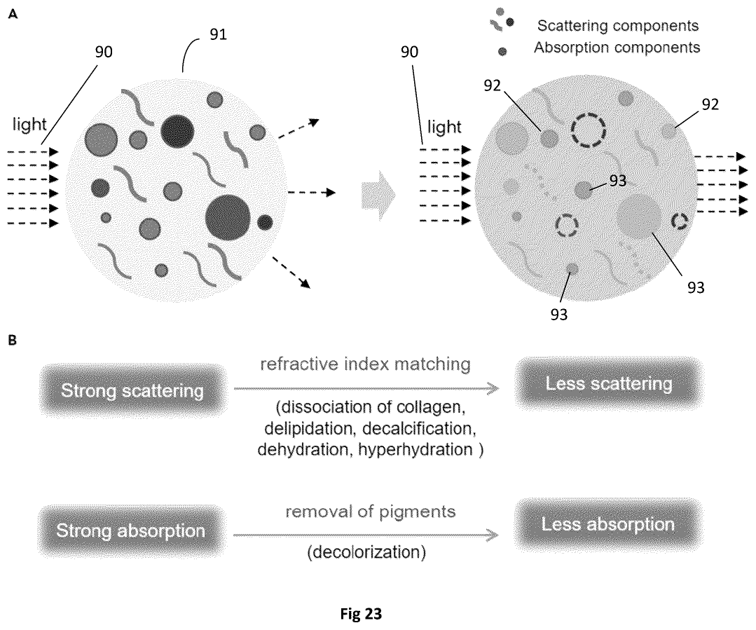

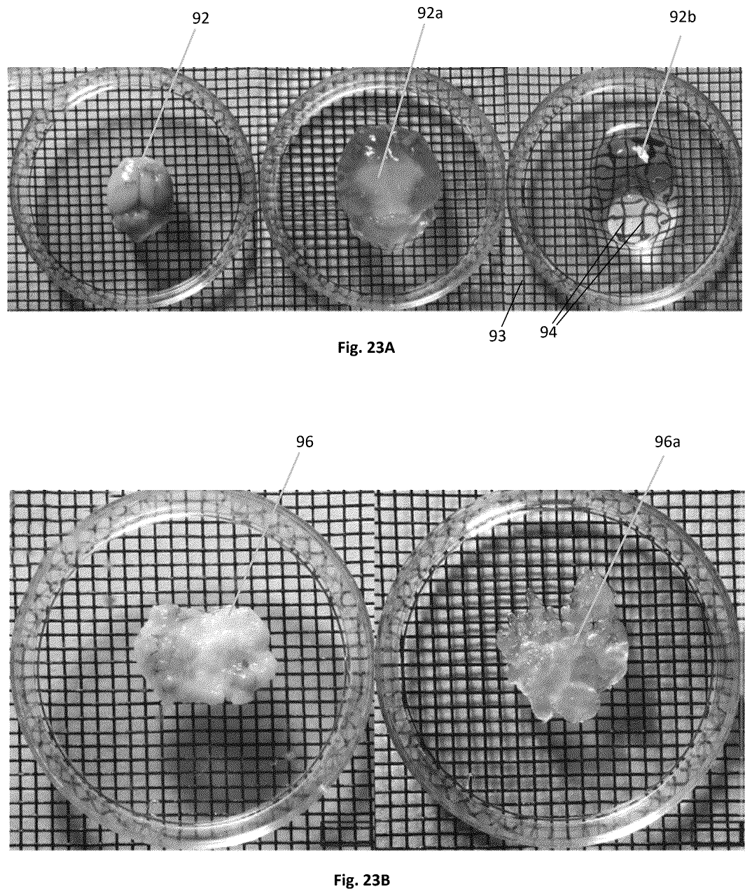

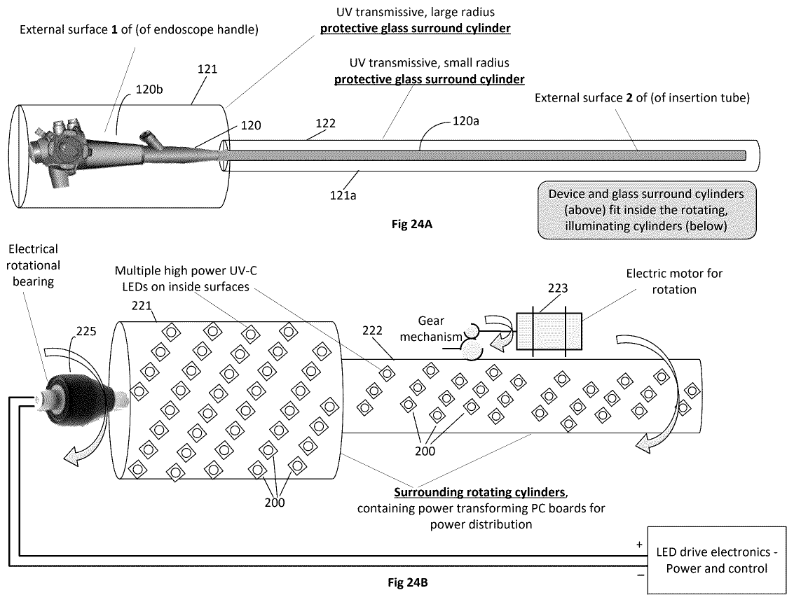

The chart in the flow chart of FIG. 23 illustrates that light (including UV light) is normally blocked from passing through tissue 91 as a result of the presence in the tissue of light scattering lipids and light absorption pigments. Refractive index matching with dissociation of collagen, delipidation, decalcification, dehydration and hyperhydration serve to lower the light scattering effect within tissues and cells. Pigment removal with decolorization serves to reduce light absorption within the tissues and cells to the extent that the tissues become essentially transparent with the tissue clearing. This has been done in vivo for direct observation of biological processes within animals and some work with humans.

FIG. 23A shows a prior art, Sung, K. et al. Simplified three-dimensional tissue clearing and incorporation of colorimetric phenotyping. Sci. Rep. 6, 30736; doi: 10.1038/srep30736 (2016), tissue clearing of a mouse brain 92 from the opaque to translucent 92a (12 days) to completely transparent 92b (19 days), wherein markings 94 on a base support 93 are visible therethrough. FIG. 9C shows a similar prior art tissue clearing of a human basal tumor 96 from opaque to translucent/transparent 96a (21 days). These studies thus validate tissue clearing in tumors of humans whereby UV light has extended depth of penetration of at least a millimeter to full tumor penetration and UV light treatment of all cancer cells in a tumor without trauma such as with x-ray or gamma radiation. In addition, tumor size is controllable without trauma and possibly to the extent of even stage 4 cancer treatment.

It is understood the procedures described in FIGS. 4-12 may be used in combination with the tissue clearing procedures in order to facilitate operation with increased UV light depth of penetration.

Judicious mapping of the tumor with controlled administration of tissue clearing chemicals effectively confines the chemical effect of tissue clearing to the tumor and some surrounding tissue (to ensure complete cancer tumor eradication). Tissue clearing is not a significant toxic procedure per se, particularly when so confined, and restoration of lipids and pigments to cleared tissues are also believed to occur over time. Confinement of the tissue clearing to a tumor (and surrounding tissue) is also an automatic barrier for prevention of spreading of tissue necrosis by the applied UV light to areas surrounding the tumor which have not been cleared because of the lack of significant depth of penetration in such areas.

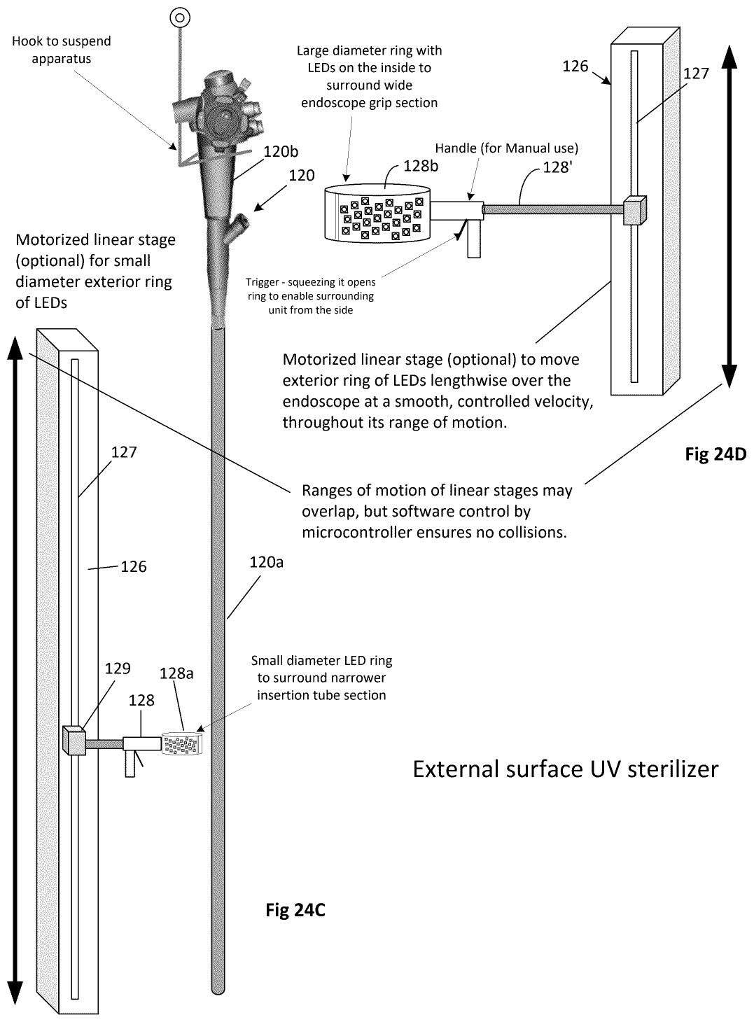

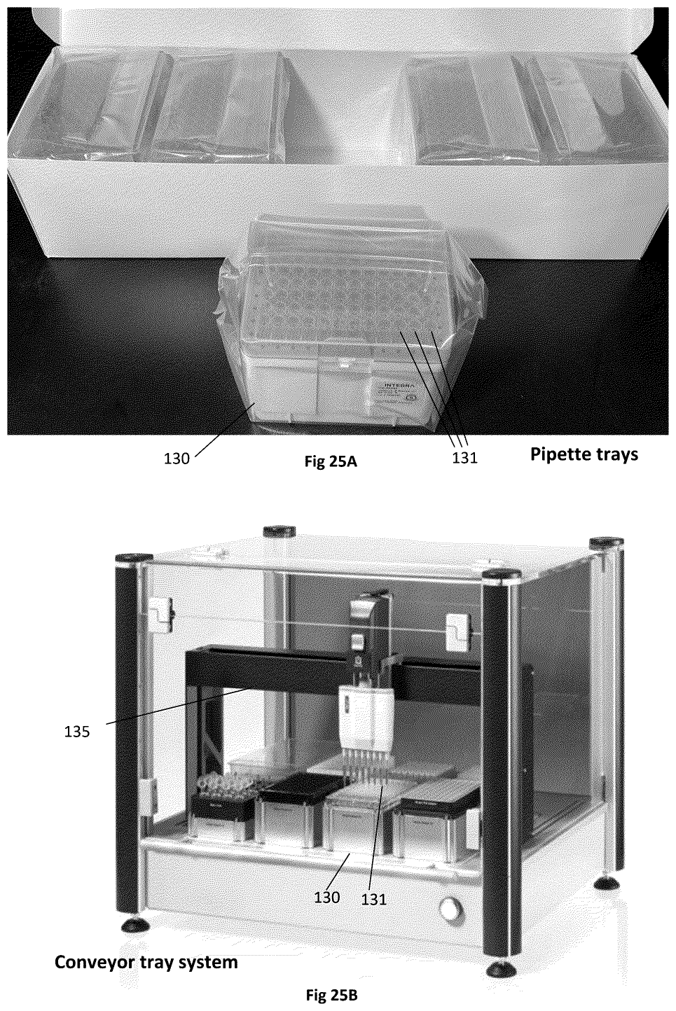

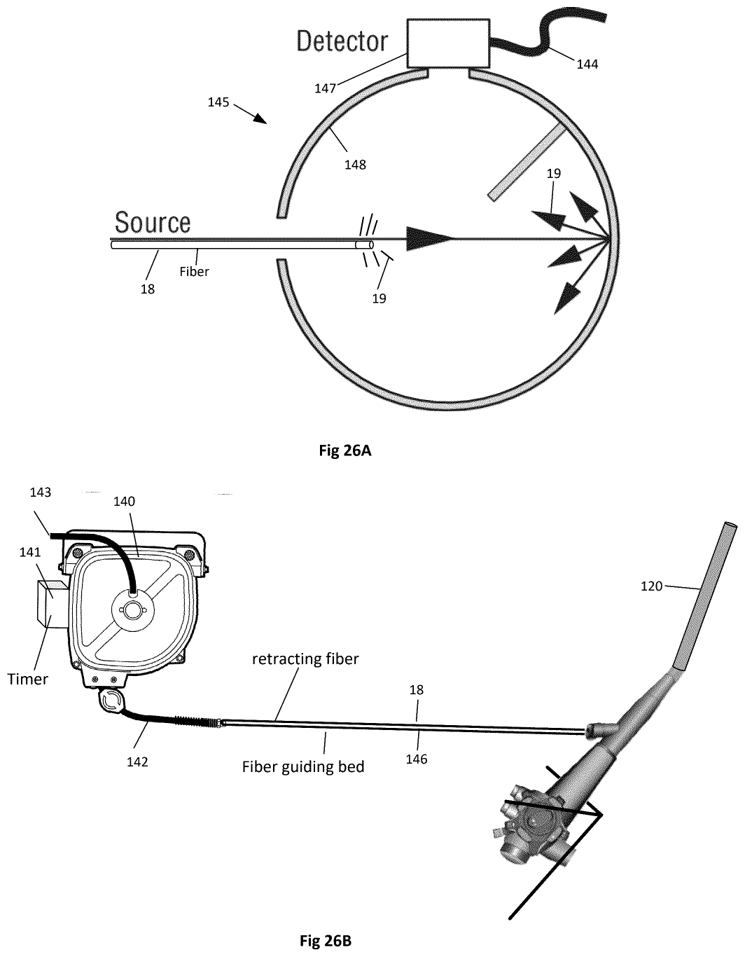

FIGS. 24A-C illustrate various embodiments wherein UV light may be utilized for rapidly sterilizing the exterior of endoscopes or other similar medical instruments. Though, as described above, full endoscope disinfection with UV light has been eschewed because of fears of endoscope polymer breakdown by the UV light, the apparatus embodiments of FIGS. 24A allow for very rapid sterilization before the UV used in the sterilization has time to affect polymers (DNA/RNA disruption in pathogens by UV is much more rapid than breakdown of polymeric bonds). FIG. 24A shows an endoscope 120 with operational handle section 120b of a first diameter and the insertion tube 120a of a lesser diameter enclosed and position locked within a transparent protective enclosure 121 having a larger diameter 121 and a smaller diameter 122.

The endoscope 120, as enclosed in protective enclosure 121, is fitted into a larger corresponding structure 221 and 222 shown in FIG. 24B with the structure 221 having an array of UV emitting LEDs on the inner surface thereof. The protective enclosure 121 is engaged with rotating element 225 powered by motor 223. The endoscope 120 is thereby completely bathed in UV (passing through protective enclosure 121) from a close distance and when calculated full sanitization is effected, the LEDs are turned off and the protective enclosure is removed from structure 221 and the endoscope is removed the protective enclosure 121. The UV light exposure time, depending on the power of the LEDs, of the endoscope can be as little as several seconds to several minutes, well under any time for the affecting of the polymeric components of the endoscope by the UV light. Automatic control time for LED activation with rotation ensures that the endoscope is not expose to UV light for any excess time above that need for sanitization.

FIGS. 24C and 24D depict another automated method for sterilization of an endoscope with UV light. In FIG. 24C endoscope 120 with small diameter insertion tube 120a and larger diameter handle 120b is suspended by hook 120′. Track element 126 has a track groove 127 of length A equal to or greater than the insertion tube 120a. Movable track element 129 controllably moves up and down the length of track groove 127 and has handle section 128 and small ring 128a (sized to closely accommodate but not touch insertion tube 120a) with UV LEDs distributed around the inner circumference thereof. In use, the track groove 127 is aligned with insertion tube 120a and the insertion tube is inserted within ring 128a. The LEDs in ring 128a are activated and the ring 128a is either manually moved with handle 128 or mechanically moved with movement of track element 129 along the length of the track groove 127 whereby every portion of insertion tube 120a is bathed with UV light for a sufficient time for the sanitization thereof.

FIG. 24D shows a similar track element 126a with track groove 127a of length B (the same as or larger than handle section 120b of the endoscope) with track element 129a, handle section 128′ and larger ring 128′ a sized to accommodate the greater diameter of handle 120B. The handle 120b of the endoscope 120 is similarly sanitized as with the procedure used with the insertion tube. Time of UV exposure should be limited to that required for sanitization and not much more to avoid any effect on polymeric integrity

FIGS. 25A and 25B illustrate an example of a manufacturing procedure requiring sanitization of large amounts of items such as laboratory pipettes, syringes or catheters (generally all of cylindrical configuration), FIG. 25A depicts an array of manufactured pipettes 131 in a processing case 130 with their respective open ends facing upward. Conveyor sanitization station 135 in FIG. 25B shows a linear placement of numerous cases 130 with arrays of pipettes as shown in FIG. 25A where the pipette cases are arranged on an assembly line in a uniform bulk configuration. At the station position shown, mass sanitization element 136 is movably positioned on track 137. Sanitization element 136 contains downwardly extending short UV transmission fibers 138 in an array and relative position and number corresponding to the pipettes in cases 130. The sanitization element 136 is moved and stopped into alignment position successively with the pipettes 131 in cases 130. The array of aligned UV transmitting short fibers are lowered en masse and inserted into the aligned pipettes 131 and UV light is transmitted from the fibers into each of the aligned pipettes for a time sufficient to effect sanitization. The fibers are withdrawn for a next batch to be sanitized with the sanitization element being moved to the next case for the same sanitization with the process being continuous, rapid and economical without need for chemicals or washing in a reliable sanitization procedure.

An automated and data-controlled biopsy channel disinfection apparatus is shown, with a disinfection protocol, in FIGS. 26A-B. As a first step, in FIG. 26A, the distal end 18″ of a UV emitting fiber 18 is tested for amount of emitting UV power by insertion into an output measuring device, integrating sphere 145 which collects power impinging on wall 148 and collection output measurement to detector 147. An alternative UV emitting laser apparatus 10 as shown in FIG. 1A has an integrated power output measuring meter positioned to measure UV power output 9a.

The output power from either the detector 147 in FIG. 26A or the power measuring meter in FIG. 1A is provided to a computer (not shown) which is also provided with the factors of fiber diameter and inner diameter of the biopsy channel for a determination of distance between the fiber and biopsy walls to be disinfected. Tables of pathogens with individual pathogen requirements for DNA/RNA disruption are contained in a database of the computer which calculates appropriate position dwell time for effective disinfection (with additional safety margin). The computer output is sent to timer 141 and retraction device 140 in FIG. 12B via input 143 for control of fiber retraction time by retraction device 140. The UV emitting fiber 18 is fully inserted into the biopsy channel 120 in FIG. 12B, connected to puller element 142 and retraction device 140 is activated to retract the fiber 18 while UV light is continuously passing through fiber 18 and out the distal end thereof with UV light 19 sanitizing the walls of the biopsy channel with continuous position UV emission and dwell time conforming to the calculated value for full disinfection. The retraction device may either be linear (with a length greater than the length of the biopsy channel on fiber guiding bed 246) such as with a relatively non-flexible fiber or spool shaped, with a flexible fiber. The process may be a single step one, or it may be repeated for greater certainty in disinfection if necessary. In any event, disinfection times are generally from about one to five minutes depending upon ascertained values of UV power and application distances. With the automation and computer control, human error and inconsistencies are minimized to ensure a more reliable disinfection especially since visual control is not directly possible. It is understood that disinfection times with UV light application are extended to include a safety buffer time period to ensure that disinfection is complete regardless of the nature of the pathogen being eradicated.

The fiber 18 may also be provided with an RFID device or the like to verify that the fiber is a genuine one with proper UV solarization resistance and UV transmission capability. The RFID device may also be configured to keep track of the number of times that the fiber has been used to transmit UV light with an operational cutoff at a predetermined point of unacceptable fiber degradation

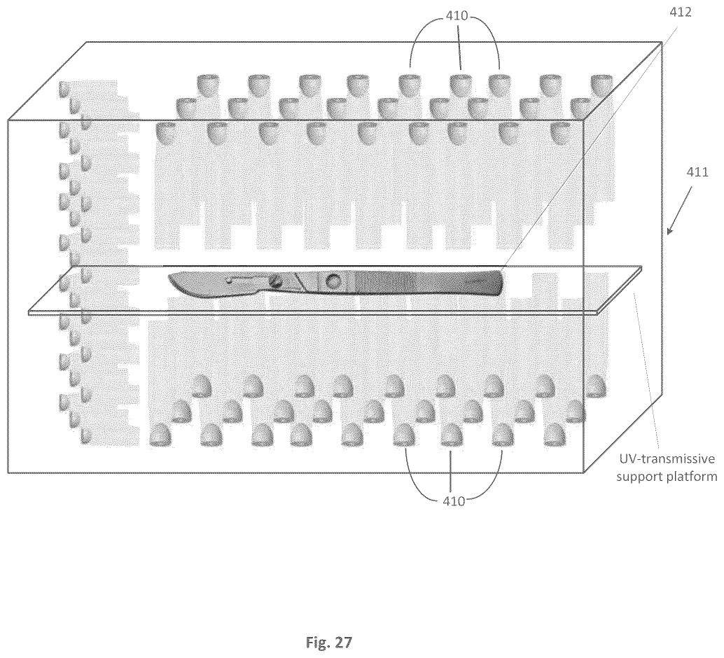

FIGS. 27 shows an embodiment where collimated lenses 410 are used to effect rapid sterilization of medical instruments particularly during surgical procedures. The enclosure or box 411 is configured for holding medical instruments such as the scalpel shown, with collimated UV lights 410 in arrays completely surrounding the scalpel 412. The scalpel rests on a UV light permeable support 413 to enable UV light to fully sterilize it from all directions. Because of different sizes and configurations of various surgical instruments, a distance, sufficient to accommodate the size of instruments to be sterilized is required from the support to the UV LED lights or the distance may be variably adjustable. The collimation lenses 410 however substantially maintain the sterilizing UV light emission power for the short distance between the light source and the sterilization sites of the instruments, rather than the normally dissipating UV light accompanied by significant sterilization power degradation.

The UV LED arrays statically surround the scalpel or the UV LED arrays rotate around the scalpel. Alternatively, the UV LED arrays move in a linear directions. Movement of the UV LED arrays helps ensure that UV light fully sterilizes the instruments including crevices and irregular surfaces of instruments. Current UV sanitization boxes without collimation which are subject to UV light dissipation, such as used for sanitization of cell phone, normally take at least ten minutes to effect acceptable sanitization. This is, however, unacceptable in a surgical setting wherein instruments need to be rapidly sterilized or exchanged. The boxes, as shown, effectively provide full sterilization in under a minute.

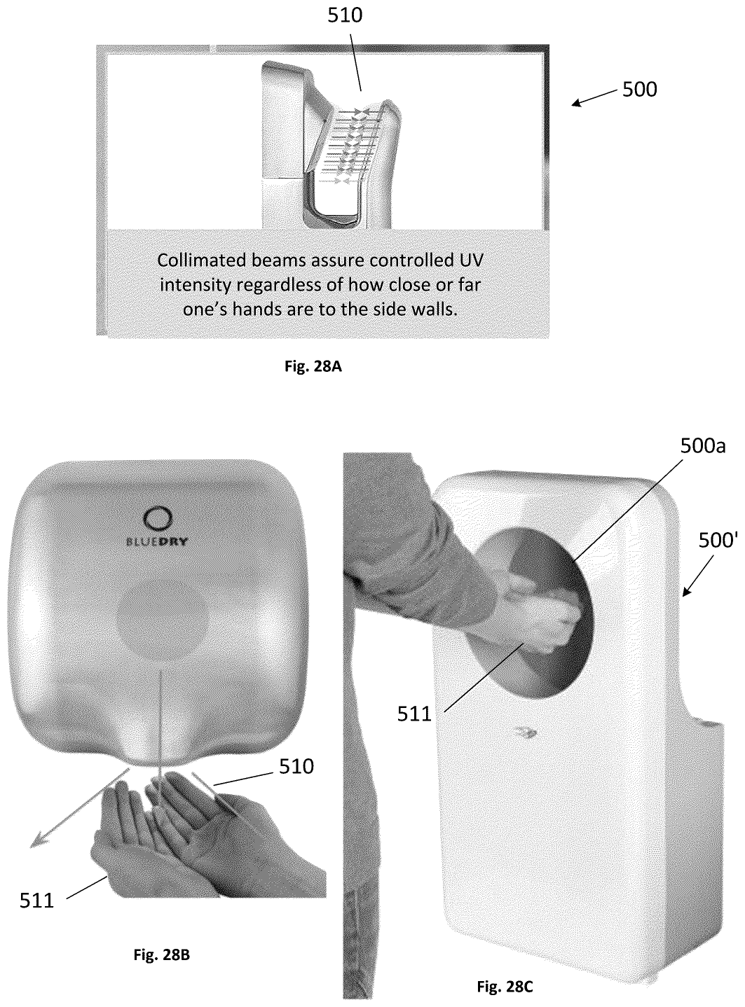

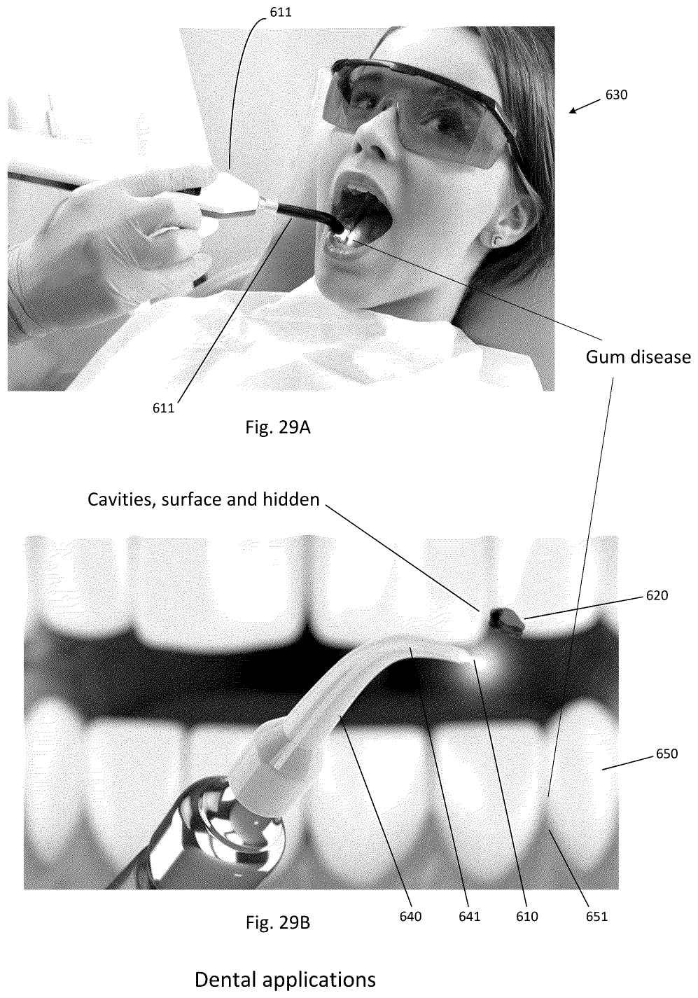

FIG. 28A is a side view of a UV light hand sanitizer 500 showing the flow of sanitizing UV light 510. Hands 511 are inserted into the slot with total hand surface impingement and sanitization. UV-C light has only a minimal surface effect (about 40 microns) whereby skin is not adversely affected but surface biological hand contaminants and pathogens are deactivated. It is understood that the use of this device is in conjunction with a hand cleaner and dryer which removes dirt which could block UV light. The sanitizer is either integrated with a hand blower or used as a separate final hand sanitization step. FIG. 28B depicts external hand sanitization with hands 511 being bathed with emitted (downward directed) UV light 510 with the hand being moved and rotated, in a manner similar to hand dryer operation. UV light is direct downward to avoid eye contact which can be irritating. FIG. 28C shows an embodiment of a sanitization device 500′ with a circular opening 500a for hand insertion with 360° placement of UV light emitting diodes.

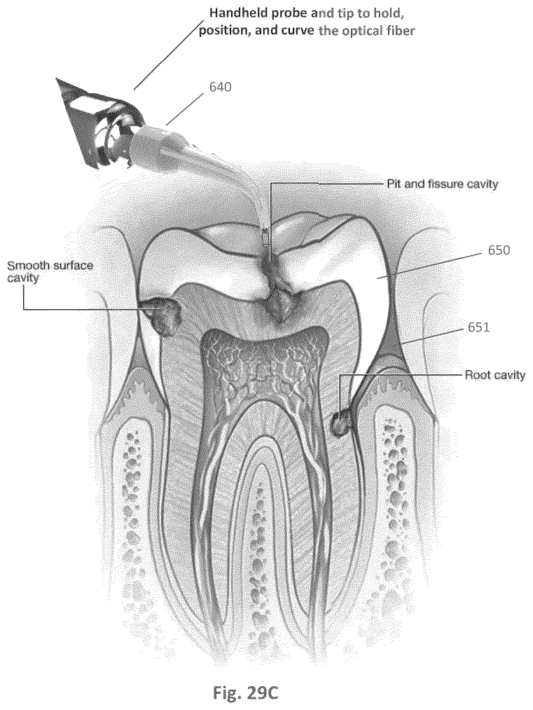

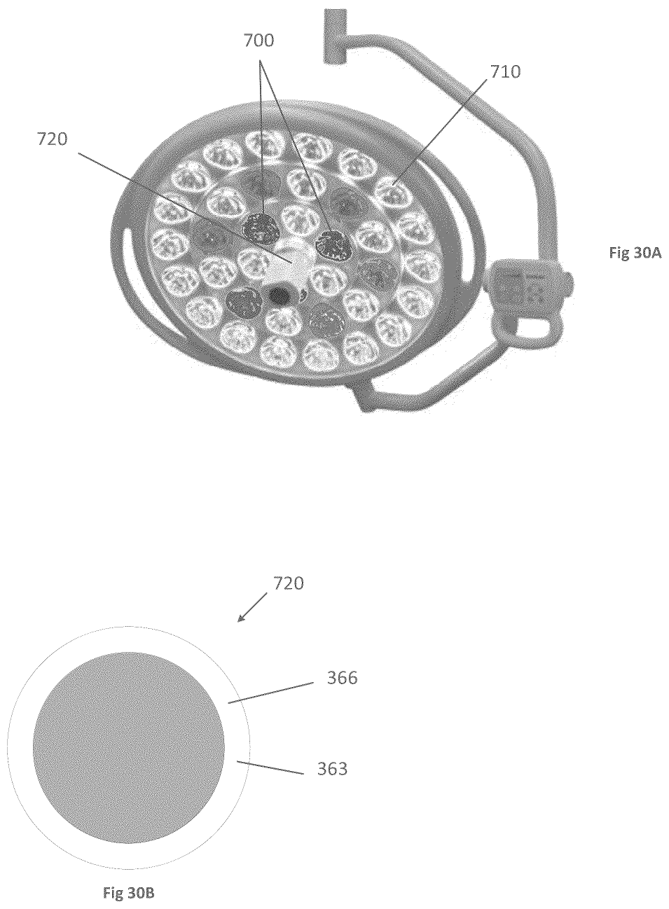

FIGS. 29A-C show a dental application of UV light in a dentist office setting 600 with UV treatment 610 of cavities and gum disease. A young patient is shown in FIG. 29A with a UV emitting instrument 640 used to treat cavities (bacterial infections) as shown in FIG. 29B. The instrument has a speculum 641 which is curved to readily reach surface areas of the teeth 650 or gums 651. FIG. 29C schematically depicts, in cross section, extension of an integrated optical fiber of small diameter (50-100 μm) which is capable of being inserted into dental fissures to reach bacterial infected sites for the disinfection thereof, including root cavities with transmitted UV light.

Dental instruments including saliva ejection system have parts that are non-disposable, no-removable and are often not autoclavable or otherwise readily sterilizable. Instruments such as the penlight or otoscope UV emitting devices described above are readily utilizable in providing focused sterilization of patient accumulated pathogens, thereby reducing occurrence of patient cross contamination.

FIGS. 30A-B show the incorporation of UV light transmitting light element 700, as modified from the penlight configuration into surgical lights 710 with camera elements 720. Head lights used by surgeons during surgical procedures which are brought closer to surgical sites may be similarly utilized to disinfect surgical sites.

It is understood that the above descriptions and examples of the invention are merely illustrative and that changes may be made to components and procedures without departing from the scope of the following claims.

What is claimed is:

1. A method for enhancing the pathogenic eradication effect of UV light of a wavelength between 200 nm to 340 nm on a pathogen infected area, the method comprising at least one of the steps of:
a. increasing effective controllable output power of a UV light source emitting device and directing the increased output power of UV light within a difficult to access pathogenic containing area with either direct placement of the UV light source within the difficult to access area or with directed transmission of UV light from the UV light source into the difficult to access area, wherein the direct or transmitted light maintains sufficient power to substantially eradicate pathogens in the pathogenic containing area, with DNA/RNA disruption,
b. placing the UV light with directed transmission of the UV light onto a surgical site before, during and optionally after a surgical procedure to maintain a substantially pathogen free environment during the surgical procedure or directing transmission of UV light through and onto an infected orifice for the disinfection thereof;
c. rendering a pathogenic infected site susceptible to significant UV light penetration with pathogen eradication thereof; or isolating a pathogenic infected site by:
i. rendering the pathogenic site more transmissive to UV light penetration;
ii. utilizing UV light pathogen eradication to form a pathogen eradicated barrier between the pathogen infected site and a non-pathogen infected site; or
iii. rendering the pathogen infected site more susceptible to the significant UV light penetration with the repeated steps of pathogenic eradication of infected areas and removal of the pathogenic eradicated areas to expose additional pathogenic infected sites to the UV light.

2. The method of claim 1, wherein the UV light source is a UV light emitting laser with relatively high UV light output power sufficient to provide pathogen eradicating power along substantially the entire length of a side emitting optical fiber optically aligned and attached to the output of the laser.

3. The method of claim 1, wherein a hand-held device is configured to provide pathogen eradicating UV light from a distance from the surgical site or infected orifice.

4. The method of claim 1, wherein the pathogen infected site is within or on a human or animal and wherein the pathogen infected site is tissue which is cleared to render it more transmissive to UV light penetration.

5. The method of claim 1 wherein UV light is collimated for a distance within which pathogens are positioned and wherein output power is maintained at such distance for the pathogen eradication.

6. The method of claim 5 wherein UV light is collimated within a hand-held device, comprising a penlight or otoscope configuration, whereby collimated light is emitted therefrom for pathogen eradication within the power maintaining collimated distance.

7. The method of claim 6, wherein the hand-held device is configured to emit light at a wavelength which causes bacteria to fluoresce to enable identifying of bacteria infected sites and to provide feedback of UV light effectiveness in eradicating the pathogens.

8. A hand-held device configured to effect the method of claim 6 comprising a UV light source, a source for emitting light at a wavelength which causes bacteria to fluoresce to a degree and extent which enables the fluorescence to be used for UV light direction for pathogen eradication and to determine effectiveness of such pathogen eradication.

9. A device for effecting the method of claim 5, comprising collimated UV light from a light source with a direction of the collimated UV light to hands positioned within the power-maintained distance for the sanitization of the hands.

10. A medical instrument disinfection enclosure for effecting the method of claim 5, comprising a stationary or moving array of collimated UV light trained on the instrument for the rapid sterilization thereof.

11. A method for providing a prophylactic prevention of any one of bladder, stomach and pancreatic cancer comprising the respective steps of emitting UV light, to eradicate possible cancer forming cells in situ, on a bladder wall, a stomach lining and a pancreatic cystic respectively with UV light of sufficient power to eradicate the possible cancer forming cells.

12. A device for effecting the method of claim 5 comprising a catheter or an endoscope having an instrument channel wherein the catheter or endoscope contains at least one UV emitting LED in the catheter or instrument channel and wherein a distal open end of the catheter comprises a collimating lens whereby UV light is emitted from the catheter or endoscope for a collimated light distance with a power sufficient to eradicate pathogens in a collimated light path.

13. A device for effecting the method of claim 5 comprising a UV light source configured to emit UV light to eradicate pathogens in a surgical site within the collimated light distance.

14. A device for effecting the method of claim 5 comprising a UV light source configured to emit UV light to eradicate pathogens in a dental site of teeth or gums within the collimated light distance.

15. A method in accordance with claim 1, wherein medical implants are sterilized in situ by introduction of UV light into a body and trained on surfaces of the medical implants with sufficient power and for a sufficient duration to sterilize the medical implant with the pathogens thereon.
